SEQUENCE LISTING (34 sequences)

SEQ ID NO: 1 - DNA, 18 bp, artificial sequence
  synthetic oligonucleotide
tggagcaagtgcctcctc

SEQ ID NO: 2 - DNA, 21 bp, artificial sequence
  synthetic oligonucleotide
atattaccacttcgattggtc

SEQ ID NO: 3 - DNA, 21 bp, artificial sequence
  synthetic oligonucleotide
tggagctggaagtgcctcctc

SEQ ID NO: 4 - DNA, 21 bp, artificial sequence
  synthetic oligonucleotide
gtgatgggagagtccgataga

SEQ ID NO: 5 - DNA, 21 bp, artificial sequence
  synthetic oligonucleotide
gtcagctggggtcgtttcatc

SEQ ID NO: 6 - DNA, 21 bp, artificial sequence
  synthetic oligonucleotide
ctggttggcctgccctatata

SEQ ID NO: 7 - DNA, 21 bp, artificial sequence
  synthetic oligonucleotide
gtcagctggggtcgtttcatc

SEQ ID NO: 8 - DNA, 21 bp, artificial sequence
  synthetic oligonucleotide
gtgaaaggatatcccaataga

SEQ ID NO: 9 - DNA, 21 bp, artificial sequence
  synthetic oligonucleotide
gtagtcccggcctgcgtgcta

SEQ ID NO: 10 - DNA, 21 bp, artificial sequence
  synthetic oligonucleotide
gaccggttacacagcctcgcc

SEQ ID NO: 11 - DNA, 21 bp, artificial sequence
  synthetic oligonucleotide
gtagtcccggcctgcgtgcta

SEQ ID NO: 12 - DNA, 21 bp, artificial sequence
  synthetic oligonucleotide
tccttgcacaatgtcaccttt

SEQ ID NO: 13 - DNA, 21 bp, artificial sequence
  synthetic oligonucleotide
ccctgccgggatcgtgacccg

SEQ ID NO: 14 - DNA, 21 bp, artificial sequence
  synthetic oligonucleotide
tcgaagccgcggctgccaaag

SEQ ID NO: 15 - DNA, 25 bp, artificial sequence
  synthetic oligonucleotide
gccagctcaccatggatgatgatat

SEQ ID NO: 16 - DNA, 25 bp, artificial sequence
  synthetic oligonucleotide
gcttgctgatccacatctgctggaa

SEQ ID NO: 17 - DNA, 21 bp, artificial sequence
  synthetic oligonucleotide
cacgacctcgtctacttcgac

SEQ ID NO: 18 - DNA, 21 bp, artificial sequence
  synthetic oligonucleotide
acagacactcgtgcagtacgc

SEQ ID NO: 19 - DNA, 22 bp, artificial sequence
  synthetic oligonucleotide
catggtggtacatgagagctac

SEQ ID NO: 20 - DNA, 22 bp, artificial sequence
  synthetic oligonucleotide
ggcaaatacaactccagctgag

SEQ ID NO: 21 - DNA, 20 bp, artificial sequence
  synthetic oligonucleotide
gagagcctccccgtccacag

SEQ ID NO: 22 - DNA, 22 bp, artificial sequence
  synthetic oligonucleotide
ctgccaggagtgtattcgcatc

SEQ ID NO: 23 - DNA, 22 bp, artificial sequence
  synthetic oligonucleotide
caggaactacgtggagatcatg

SEQ ID NO: 24 - DNA, 22 bp, artificial sequence
  synhetic oligonucleotide
ccatcccaccaaactcgatgtc

SEQ ID NO: 25 - DNA, 23 bp, artificial sequence
  synthetic oligonucleotide
gaactttcctccaacttcatggc

SEQ ID NO: 26 - DNA, 21 bp, artificial sequence
  synthetic oligonucleotide
catttccattttacagaccgg

SEQ ID NO: 27 - DNA, 21 bp, artificial sequence
  synthetic oligonucleotide
ggtgagccagcactgcaagag

SEQ ID NO: 28 - DNA, 25 bp, artificial sequence
  synthetic oligonucleotide
cctaaaagtgaaatggtttcgatcg

SEQ ID NO: 29 - DNA, 20 bp, artificial sequence
  synthetic oligonucleotide
acacgtccaacgccagcatg

SEQ ID NO: 30 - DNA, 22 bp, artificial sequence
  synthetic oligonucleotide
acgagtcatgttgtagccgatg

SEQ ID NO: 31 - DNA, 20 bp, artificial sequence
  synthetic oligonucleotide
cttcacactccccacacaga

SEQ ID NO: 32 - DNA, 20 bp, artificial sequence
  synthetic oligonucleotide
tcctgtttggtgggcttctg

SEQ ID NO: 33 - DNA, 25 bp, artificial sequence
  synthetic oligonucleotide
gccagctcaccatggatgatgatat

SEQ ID NO: 34 - DNA, 25 bp, artificial sequence
  synthetic oligonucleotide
gcttgctgatccacatctgctggaa

(19) United States
(12) Patent Application Publication
(10) Pub. No.: US 2009/0156477 A1
(43) Pub. Date: Jun. 18, 2009

(54) Compositions and Methods for Regulating Inflammatory Responses

(75) Inventors: David A. Berry, Brookline, MA (US); Chi-Pong Kwan, Framingham, MA (US); Robert S. Langer, Newton, MA (US); Ram Sasisekharan, Bedford, MA (US)

Correspondence Address: WOLF GREENFIELD & SACKS, P.C., 600 ATLANTIC AVENUE, BOSTON, MA, 02210-2206, US

(73) Assignee: Massachusetts Institute of Technology, Cambridge, MA (US)

(21) Appl. No.: 11/887,561

(22) PCT Filed: Mar. 29, 2006
(86) PCT No.: PCT/US2006/011680

§ 371 Date: Jan. 5, 2009

Related U.S. Application Data
(60) Provisional application No. 60/666,745, filed on Mar. 29, 2005.

(51) Int. Cl. A61K 38/18; A61P 29/00

(52) U.S. Cl. 514/12

(57) ABSTRACT

This invention relates, in part, to compositions and methods for the regulation of inflammatory responses. Specifically, the invention relates, in part, to compositions of and methods for using fibroblast growth factor (FGF) proteins, proteoglycans (e.g., syndecans), agents that modulate proteoglycans and agents that affect Wnt signaling. The invention also provides compositions and methods for treating subjects with undesired inflammatory activity and/or diseases associated therewith. The invention further provides, in part, compositions and methods for disrupting intercellular junctions with FGFs, such as for the enhanced delivery of therapeutic agents.

### RELATED APPLICATIONS

This application claims priority under 35 U.S.C. §119 from U.S. provisional application Ser. No. 60/666,745, filed Mar. 29, 2005. The entire contents of which is herein incorporated by reference.

### GOVERNMENT SUPPORT

Aspects of the invention may have been made using funding from National Institutes of Health Grant numbers EB-00244, CA-52857, HL-59966 and CA-90940. Accordingly, the government may have rights in the invention.

### FIELD OF THE INVENTION

This invention relates, in part, to compositions and methods for the regulation of inflammatory responses. Specifically, the invention relates, in part, to compositions of and methods for using fibroblast growth factor (FGF) proteins, proteoglycans (e.g., syndecans), agents that modulate proteoglycans and agents that affect Wnt signaling. The invention also provides compositions and methods for treating subjects with undesired inflammatory activity and/or diseases associated therewith. The invention further provides, in part, compositions and methods for disrupting intercellular junctions with FGFs.

### BACKGROUND OF THE INVENTION

The FGF family consists of at least 23 members, all of which bind to the heparin/heparan sulfate-like glycosaminoglycan (HSGAG) component of heparan sulfate proteoglycans (HSPGs) and to at least one of five high affinity cell surface FGF receptors (FGFRs) [8-11]. While FGFs are best known for their role in angiogenesis [12], FGF2, FGF7, FGF10 and FGF20, have each been implicated as having a potential role in regulating the severity and progression of inflammatory bowel disease (IBD) [5,12-13]. Angiogenic factors including FGFs and vascular endothelial growth factor (VEGF) not only determine the susceptibility of the IBD colonic mucosa to injury but also increase blood vessel formation and bowel wall thickening [16-18].

FGFs are released by colonic intraepithelial γδ T cells, as well as by fibroblasts and smooth muscle cells of the lamina propria, to maintain and repair the colonic epithelium [15,19,20]. In addition to their angiogenic functions, FGFs stimulate the proliferation and migration of intestinal epithelial cells, important for wound healing [21-23]. FGF2 and FGF7 specifically promote intestinal epithelial cell growth and wound healing [24]. FGF2 can accelerate gastrointestinal wound healing and resist insults that lead to colonic inflammation [21,23]. FGF7 is found in the colonic mucosa of IBD patients and reduces the inflammatory response [25,26]. The cellular mediated effects of these ligands, however, have been associated with FGFR3b, in cells such as Caco-2 cells [27]. FGF1 and FGF9 bind and promote a downstream response through FGFR3b [28]. FGF2 promotes cellular responses through FGFR1c, FGFR3c and FGFR4, while FGF7 exclusively requires FGFR2b [29]. The mechanism by which FGF2 and FGF7 produce their effects in the colon, however, is not clear. In addition, although various FGF family members reduce the severity of colitis and promoted healing in IBD both in vitro and in vivo [30,31], little therapeutic benefit has been observed in human studies [32].

### SUMMARY OF THE INVENTION

The inventions relates, in part, to compositions and methods, that are useful for the regulation of inflammatory responses and/or the treatment of diseases associated therewith. The compositions can, for example, comprise FGF proteins, proteoglycans (e.g., syndecans), agents that modulate proteoglycans, agents that modulate Wnt signaling or some combination thereof. The invention also relates, in part, to compositions and methods for disrupting intercellular junctions with FGFs. Such compositions and methods can be used to enhance the delivery of therapeutic agents.

Therefore, in one aspect of the invention, a method for altering an inflammatory response is provided. In one embodiment, the method includes the step of contacting one or more cells affected by an inflammatory response with a composition comprising fibroblast growth factor-1 (FGF1) and at least one other fibroblast growth factor (FGF), wherein the composition is in an amount effective to alter the inflammatory response. In another embodiment, the at least one other FGF is FGF2, FGF7, FGF10 or FGF20. In yet another embodiment, the at least one other FGF is FGF2, FGF7 or both. In still another embodiment, the FGF1 is in a multimeric (e.g., dimeric) form. In a further embodiment, the at least one other FGF is in a dimeric form. In yet another embodiment, the at least one other FGF in dimeric form is FGF2 in dimeric form. In a further embodiment, the FGF1 and the at least one other FGF are both in a dimeric form. In still a further embodiment, the FGF2 in dimeric form is a FGF2 homodimer.

In another aspect of the invention, a method for altering an inflammatory response by contacting one or more cells affected by the inflammatory response with a composition comprising a stabilized FGF multimer (e.g., dimer) is provided. In one embodiment, the composition is in an amount effective to alter the inflammatory response. In another embodiment, the inflammatory response is not associated with a wound, a scar, an ulcerating disease, inflammatory neuropathy or chronic inflammatory demyelinating polyradiculoneuropathy. In still another embodiment, the stabilized FGF dimer is a stabilized FGF2 dimer. In a further embodiment, the stabilized FGF dimer is a stabilized FGF homodimer. In yet a further embodiment, the stabilized FGF homodimer is a stabilized FGF2 homodimer.

In a further aspect of the invention, a method for altering an inflammatory response in a subject by administering to the subject a proteoglycan agent (e.g., syndecan agent) is provided. In one embodiment, the proteoglycan agent (e.g., syndecan agent) is in an amount effective to alter the inflammatory response. In another embodiment, the proteoglycan agent (e.g., syndecan agent) is not TMB. In still another embodiment, the proteoglycan agent is a proteoglycan, such as a HSPG, CSPG or a KSPG. In another embodiment, the proteoglycan agent (e.g., syndecan agent) is a syndecan. In still another embodiment, the syndecan is syndecan-1, syndecan-2, syndecan-3, syndecan-4 or some combination thereof. In a further embodiment, the syndecan agent is a combination of syndecan-1 and syndecan-4. In a further embodiment, the proteoglycan agent (e.g., syndecan agent) is in an amount effective to promote proteoglycan (e.g., syndecan) shedding. In yet another embodiment, the proteoglycan agent (e.g., syndecan agent) is an agent that promotes proteoglycan (e.g., syndecan) shedding. In one embodiment, the agent that promotes proteoglycan shedding is an agent that promotes syndecan shedding, and the agent that promotes syndecan shedding is matrilysin, EGF or PIF. In another embodiment, the proteoglycan agent (e.g., syndecan agent) is an agent that increases the production of the proteoglycan (e.g., syndecan). In yet another embodiment the proteoglycan is a syndecan, and the syndecan with increased production is syndecan-1 or syndecan-4.

In a further aspect of the invention, a method for altering an inflammatory response by contacting one or more cells affected by the inflammatory response with a composition comprising an agent that alters Wnt signaling is provided. In one embodiment, the composition is in an amount effective to alter the inflammatory response. In another embodiment, the agent the alters Wnt signaling is an activator of Wnt signaling. In a further embodiment, the activator is Disheveled or an inhibitor (e.g., an antibody) to Dikkopf. In yet a further embodiment, the agent that alters Wnt signaling is an inhibitor of Wnt signaling. In another embodiment, the inhibitor is Dikkopf, an inhibitor (e.g., an antibody) to Frizzled or an inhibitor (e.g., antibody) to Disheveled. In yet a further embodiment, the agent that alters Wnt signaling is a protein associated with Wnt signaling, such as Wnt1, Wnt3a, Wnt5 or Wnt10.

In still a further aspect of the invention, a method for altering an inflammatory response by modulation (e.g., with antibodies) of beta-1 integrin, beta-3 integrin, alpha-2 integrin, alpha-4 integrin or alpha-5 integrin is provided. Compositions of such modulators are also provided.

In another embodiment, the one or more cells affected by the inflammatory response are in a subject, and the composition is administered to the subject.

In still another embodiment, the subject has a disease associated with an improper immune response. In one embodiment, the disease associated with an improper immune response is an inflammatory bowel disease, autoimmune disease, a chronic disease with bouts of acute inflammation, Lyme disease, tuberculosis or multiple myeloma. In another embodiment, the inflammatory bowel disease is ulcerative colitis or Crohn's disease. In yet a further embodiment, the subject has exuberant granulomas or keloids. In a further embodiment, the subject is in need of wound healing or scar reduction. In another embodiment, the subject is in need of cell proliferation or angiogenesis. In one embodiment, the subject has an ulcer, such as a diabetic ulcer.

Compositions of the agents described herein are also provided.

In one aspect of the invention, a composition comprising FGF1 and at least one other FGF is provided. In one embodiment, the FGF1 and the at least one other FGF are in an amount effective to alter an inflammatory response. In still another embodiment, the FGF1 and the at least one other FGF are in an amount effective to treat a disease associated with an improper immune response. In another embodiment, the at least one other FGF is FGF2, FGF7, FGF10 or FGF20. In a further embodiment, the at least one other FGF is FGF2, FGF7 or both. In yet a further embodiment, the FGF1 is in a multimeric (e.g., dimeric) form. In another embodiment, the at least one other FGF is in a dimeric form. In a further embodiment, the FGF1 and the at least one other FGF are both in a dimeric form. In another embodiment, the at least one other FGF in dimeric form is FGF2 in dimeric form. In a further embodiment, the FGF2 in dimeric form is a FGF2 homodimer.

In another aspect of the invention, a composition comprising a stabilized FGF multimer (e.g., dimer) is provided. In one embodiment, the composition further comprises at least one additional therapeutic agent and/or a pharmaceutically acceptable carrier. In another embodiment, the pharmaceutically acceptable carrier is sterile. In another embodiment, the at least one additional therapeutic agent is an anti-inflammatory agent, an anti-cancer agent or an agent for treating an immunologic disorder. In a further embodiment, the stabilized FGF dimer and at least one additional therapeutic agent are in an amount effective to alter an inflammatory response. In another embodiment, the stabilized FGF dimer is a stabilized FGF2 dimer. In a further embodiment, the stabilized FGF2 dimer is a stabilized FGF2 homodimer.

In yet another aspect of the invention, a composition comprising a proteoglycan agent (e.g., a syndecan agent) is provided. In one embodiment, the composition further comprises a pharmaceutically acceptable carrier. In another embodiment, the pharmaceutically acceptable carrier is sterile. In another embodiment, the proteoglycan agent (e.g., a syndecan agent) is not TMB. In still another embodiment, the proteoglycan agent (e.g., a syndecan agent) is in an amount effective to alter an inflammatory response. In a further embodiment, the proteoglycan agent (e.g., a syndecan agent) is in an amount effective to promote proteoglycan (e.g., syndecan) shedding. In one embodiment, the proteoglycan is a syndecan. In another embodiment, the syndecan is syndecan-1, syndecan-2, syndecan-3, syndecan-4 or some combination thereof. In a further embodiment, the syndecan is a combination of syndecan-1 and syndecan-4. In a further embodiment, the proteoglycan agent (e.g., syndecan agent) is in an amount effective to promote proteoglycan (e.g., syndecan) shedding. In yet another embodiment, the proteoglycan agent (e.g., syndecan agent) is an agent that promotes proteoglycan (e.g., syndecan) shedding. In one embodiment, the agent that promotes proteoglycan shedding is an agent that promotes syndecan shedding, and the agent that promotes syndecan shedding is matrilysin, EGF or PIF. In another embodiment, the proteoglycan agent (e.g., syndecan agent) is an agent that increases the production of the proteoglycan (e.g., syndecan). In yet another embodiment the proteoglycan is a syndecan, and the syndecan with increased production is syndecan-1 or syndecan-4.

In another aspect of the invention, a composition that comprises an agent that alters Wnt signaling is provided. In one embodiment, the agent that alters Wnt signaling is in an amount effective to alter an inflammatory response. In another embodiment, the agent that alters Wnt signaling is an activator of Wnt signaling. In a further embodiment, the activator is Disheveled or an inhibitor (e.g., an antibody) to Dikkopf. In yet a further embodiment, the agent that alters Wnt signaling is an inhibitor of Wnt signaling. In another embodiment, the inhibitor is Dikkopf, an inhibitor (e.g., an antibody) to Frizzled or an inhibitor (e.g., antibody) to Disheveled. In yet a further embodiment, the agent that alters Wnt signaling is a protein associated with Wnt signaling, such as Wnt1, Wnt3a, Wnt5 or Wnt10.

In another embodiment, the compositions described herein further comprise a pharmaceutically acceptable carrier. In another embodiment, the pharmaceutically acceptable carrier is sterile.

In yet another embodiment, the compositions described herein further comprise an additional therapeutic agent. In still another embodiment, the methods provided further comprise administering an additional therapeutic agent. In one embodiment, the additional therapeutic agent is an anti-inflammatory agent, an anti-ulcer agent, an anti-cancer agent, an agent for treating an immunologic disorder or insulin. In still another embodiment, the additional therapeutic agent is an agent for treating IBD.

The compositions provided herein, in some embodiments, can be used in the treatment of any of the diseases described herein. In other embodiments, the compositions provided can be used to treat a subject who has exuberant granulomas or keloids. In a further embodiment, the compositions provided can be used to treat a subject in need of wound healing or scar reduction. In another embodiment, the compositions provided can be used to treat a subject in need of cell proliferation or angiogenesis. In a further embodiment, the compositions provided can be used to promote cell proliferation or angiogenesis. In another embodiment, the composition provided can be used to treat a subject who has an ulcer, such as a diabetic ulcer.

In yet another aspect of the invention, a method for transiently disrupting intercellular junctions in a subject is provided. In one embodiment, the method includes the step of administering to a subject a composition comprising a FGF in an amount effective to transiently disrupt intercellular junctions. In one embodiment, the FGF is FGF2, FGF7, FGF10 or FGF20. In another embodiment, the FGF is in a multimeric (e.g., dimeric) form. In still another embodiment, the FGF in dimeric form is FGF2 in dimeric form. In still another embodiment, the FGF2 in dimeric form is a FGF2 homodimer. In a further embodiment, the method further comprises administering a therapeutic agent. In another embodiment, the subject has a disease associated with an improper immune response. In one embodiment, the therapeutic agent is an anti-inflammatory agent, an anti-ulcer agent, an anti-cancer agent, an agent for treating an immunologic disorder or insulin. In yet another embodiment, the therapeutic agent is an agent for treating IBD. In still another embodiment, the therapeutic agent is administered prior to, concomitantly with or subsequent to the administration of the composition comprising FGF. In yet a further embodiment, the therapeutic agent and FGF are linked. In one embodiment, the composition comprising FGF is administered rectally, orally or by pulmonary, buccal or sublingual administration. In one embodiment, the delivery of the therapeutic agent is enhanced. In another embodiment, the delivery of the therapeutic agent through a cell barrier is enhanced.

In another aspect of the invention, uses of the compositions provided for the preparation of a medicament are also provided. In one embodiment, the medicament is for modulating (e.g., increasing) an activity of a FGF, VEGF family member or both. In another embodiment, the medicament is for treating a disease associated with an improper immune response. In yet another embodiment, the medicament is for treating a subject who has exuberant granulomas or keloids. In a further embodiment, the medicament is for treating a subject in need of wound healing or scar reduction. In still a further embodiment, the medicament is for treating a subject in need of cell proliferation or angiogenesis. In one embodiment, the medicament is for treating a subject with an ulcer, such as a diabetic ulcer.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE FIGURES

FIG. 1 illustrates that FGF1 causes reductions in TER. TER was measured in Caco-2 (FIG. 1A) and T84 (FIG. 1B) cells. Cells were treated with PBS or FGF1 on days 10 and 12. Data are presented as resistance (in ohms) averaged over three experiments. * denotes p<0.05 for FGF1 compared to PBS.

FIG. 2 illustrates that FGF1 is necessary for TER reductions induced by FGF2 and FGF7. TER was measured in Caco-2 (FIG. 2A and FIG. 2C) and T84 (FIG. 2B and FIG. 2D) cells supplemented with 200 μM TM (FIG. 2A and FIG. 2B) or with 200 μM TM and 100 ng/ml FGF1 (FIG. 2B and FIG. 2D). Data are presented as resistance (in ohms) averaged over three experiments. TM was added on days 8, 10 and 12. FGF1, FGF2 and FGF7 were added on days 10 and 12. * denotes p<0.05 for FGF1 or FGF2 compared to PBS. † denotes p<0.05 for FGF7 compared to PBS.

FIG. 3 demonstrates that FGF2 and FGF7 induce membrane ruffling in Caco-2 and T84 cells. Caco-2 and T84 cells stained for occludin were visualized by fluorescence microscopy. Scale bars represent 10 μm. Arrows illustrate regions of membrane ruffling.

FIG. 4 demonstrates that Caco-2 and T84 cells express FGFR3b. RT-PCR on Caco-2 and T84 cells was performed using primers specific to FGFR isoforms (1b, 1c, 2b, 2c, 3b, 3c and 4) and to β-actin (Act), the positive control (FIG. 4A). FACS was performed on Caco-2 and T84 cells (FIG. 4B). Cells were stained using a rabbit anti-human primary antibody to FGFR3 and a goat anti-rabbit antibody conjugated to FITC secondary antibody. Data are presented as the percentage of total cells that exhibited positive expression. Immunohistochemistry for FGFR3 was performed on Caco-2 and T84 cells (FIG. 4C). Cells were stained using a rabbit anti-human FGFR3 primary antibody and a goat anti-rabbit FITC-conjugated secondary antibody. Scale bars are as indicated.

FIG. 5 illustrates that FGF2 and FGF7 induce cellular responses in Caco-2 and T84 cells. The ability of FGF2 and FGF7 to inhibit TER increases was determined three days after the addition of ligand (FIG. 5A). Data are presented as the percent reduction in TER compared to PBS treated cells over the same time period. * denotes p<0.05 for the experimental point compared to the PBS control. Caco-2 and T84 cells were plated at 5×10⁴ cells/well (FIG. 5B). Cells were treated with PBS, FGF2 or FGF7, and whole cell number was determined after three days. Data are presented as percent reduction in whole cell number controlled to the PBS control. * denotes p<0.05 for the experimental point compared to the PBS control. Caco-2 monolayers were treated with PBS, FGF2 or FGF7, and the reduction in TER was determined over 48 hours (FIG. 5C). Data are presented as the percent of initial TER over time. * denotes p<0.05 for FGF2 compared to the PBS control. † denotes p<0.05 for FGF7 compared to the PBS control. Caco-2 (FIG. 5D) and T84 (FIG. 5E) monolayers were treated with PBS, FGF2 or FGF7 on days 0 and 2, and TER was measured over time. * denotes p<0.05 for FGF2 compared to the PBS control. † denotes p<0.05 for FGF7 compared to the PBS control.

FIG. 6 demonstrates that FGFR3 is necessary for FGF2 and FGF7 to reduce TER. TER was measured in Caco-2 (FIGS. 6A, 6C and 6E) and T84 (FIGS. 6B, 6D and 6F) cells after administration of PBS, FGF2 or FGF7 along with anti-FGFR3 antibodies (FIGS. 6A and 6B), anti-FGFR1 antibodies (FIGS. 6C and 6D) or LY294002 (FIGS. 6E and 6F). Data are presented as resistance (in ohms) as measured on day 0 and day 2, and on day 5. Antibodies, kinase inhibitors, FGF2 and FGF7 were added at the same time on both day 0 and day 2. * denotes p<0.05 for FGF2 compared to the PBS control. † denotes p<0.05 for FGF7 compared to the PBS control.

FIG. 7 illustrates that FGF1 elicits cellular responses in Caco-2 and T84 cells. Caco-2 monolayers were treated with PBS or FGF1, and the reduction in TER was determined over 48 hours (FIG. 7A). Data are presented as the percent reduction in TER over time. * denotes p<0.05 for FGF1 compared to the PBS control. The ability of FGF1 to reduce TER increases was determined 72 hours after the addition of ligand (FIG. 7B). Data are presented as the percent reduction in TER compared to PBS treated cells over the same time period. * denotes p<0.05 for the experimental point compared to the PBS control, Caco-2 and T84 cells were plated at 5×10⁴ cells/well (FIG. 7C). Cells were treated with PBS or FGF1, and whole cell number was determined after three days. Data are presented as whole cell number/100.

FIG. 8 illustrates that cell surface HSGAGs are necessary for FGF-mediated changes in TER. TER was measured in Caco-2 (FIG. 8A) and T84 (FIG. 8B) cells supplemented with 50 mM sodium chlorate. Sodium chlorate was added 24 hours prior to day 0, on day 0 and on day 2. PBS and FGFs were added on day 0 and on day 2. Data are presented as resistance (in ohms) averaged over three experiments. * denotes p<0.05 for FGF1 compared to PBS. † denotes p<0.05 for FGF2 compared to PBS. § denotes p<0.05 for FGF7 compared to PBS.

FIG. 9 demonstrates that FGF1 is necessary for FGF2 and FGF7 reductions in TER. PBS, FGF2 or FGF7 was added to Caco-2 monolayers pretreated with TM, and the reduction in TER was determined over 48 hours (FIG. 9A). Data are presented as the percent reduction in TER over time. * denotes p<0.05 for FGF2 compared to the PBS control. † denotes p<0.05 for FGF7 compared to PBS. PBS, FGF2 or FGF7 was added to Caco-2 monolayers pretreated with TM and FGF1, and the reduction in TER was determined over 48 hours (FIG. 9B). Data are presented as the percent reduction in TER over time. * denotes p<0.05 for FGF2 compared to the PBS control. † denotes p<0.05 for FGF7 compared to PBS. Immunohistochemistry for FGF1 was performed on untreated Caco-2 cells (FIG. 9C) and Caco-2 cells treated with 200 μM TM for 24 hours (FIG. 9D). TER was measured in T84 cells pretreated with TM (FIG. 9E) or both TM and FGF1 (FIG. 9F). TM and FGF1 were added 24 hours prior to day 0, day 0 and day 2. PBS, FGF2 and FGF7 were added at day 0 and day 2. Data are presented as resistance (in ohms) averaged over three experiments. * denotes p<0.05 for FGF2 compared to PBS. † denotes p<0.05 for FGF7 compared to PBS.

FIG. 10 demonstrates that FGFs reduce TER in Caco-2 monolayers. Caco-2 cell monolayers were treated with PBS, FGF1, FGF2 or FGF7, and TER was measured over 48 hours. * denotes p<0.05 for FGF1 treated cells compared to PBS treated cells. † denotes p <0.05 for FGF2 treated cells compared to PBS treated cells. § denotes p<0.05 for FGF7 treated cells compared to PBS treated cells.

FIG. 11 illustrates that FGF1 is necessary for FGF2 and FGF7 reductions in TER. Caco-2 cell monolayers were pretreated with TM. Cells were treated with PBS, FGF1, FGF2 or FGF7, and TER was measured for 48 hours (FIG. 11A). * denotes p<0.05 for FGF1 treated cells compared to PBS treated cells. Cells were treated with FGF1 along with either FGF2 or FGF7 (FIG. 11B). Control cells were treated with TM, but not FGF1. TER was measured over 48 hours. † denotes p<0.05 for FGF2 treated cells compared to PBS treated cells. § denotes p<0.05 for FGF7 treated cells compared to PBS treated cells.

FIG. 12 shows that FGF2 and FGF7 affect ZO-1 as well as actin stress fiber formation. Caco-2 cell monolayers were treated with PBS, FGF2 or FGF7. Immunohistochemistry was performed for ZO-1 and f-actin. Images were captured by confocal microscopy. Scale bars are as indicated.

FIG. 13 illustrates that cell surface HSGAGs are necessary for FGF2 and FGF7 reductions in TER. Caco-2 cell monolayers were pretreated with sodium chlorate. Cells were treated with PBS, FGF1, FGF2 or FGF7, and TER was measured over 48 hours (FIG. 13A). Cells were treated with heparin as well as PBS, FGF1, FGF2 or FGF7, and TER was measured over 48 hours (FIG. 13B). * denotes p<0.05 for FGF1 treated cells compared to PBS treated cells.

FIG. 14 shows that cell surface HSPGs are necessary for FGF2 and FGF7 reductions in TER. Caco-2 cell monolayers were pretreated with MMP-2, and subsequently, PBS, FGF1, FGF2 or FGF7 was added. TER was measured over 48 hours. * denotes p<0.05 for FGF1 treated cells compared to PBS treated cells.

FIG. 15 shows that FGFs alter syndecan-1 localization. Caco-2 cell monolayers were treated with PBS, FGF1, FGF2 or FGF7 for 1 or 24 hours (FIG. 15A). Immunohistochemistry was performed for syndecan-1 and DAPI. Images were captured by fluorescence microscopy. Scale bars are as indicated. Caco-2 cell monolayers were treated with PBS (left) or FGF1 (middle and right) for 4 hours, and images were captured by fluorescence microscopy (FIG. 15B). Immunohistochemistry was performed for syndecan-1, caveolin-1 and DAPI. The left image shows an overlay of staining for syndecan-1, caveolin-1 and DAPI after PBS treatment. The middle panel represents staining for caveolin-1 and DAPI after FGF1 treatment. The right image shows an overlay of staining for syndecan-1, caveolin-1 and DAPI after FGF1 treatment. Scale bars are as indicated. Caco-2 monolayers were treated with FGF1 for 24 hours, and immunohistochemistry was performed for FLAER, syndecan-1 and DAPI (FIG. 15C). Images were captured by fluorescence microscopy. Scale bars are as indicated.

FIG. 16 illustrates that Wnt signaling is necessary for FGF2 and FGF7 reductions in TER. Caco-2 cell monolayers were treated with anti-Fz1 antibodies, as well as PBS, FGF1, FGF2 or FGF7 (FIG. 16A). TER was measured over 48 hours. RT-PCR was performed on Caco-2 cells after treatment with PBS, FGF1, FGF2 or FGF7 (FIG. 16B).

FIG. 17 shows that FGFs affect the Wnt signaling cascade. Caco-2 cell monolayers were treated with PBS, FGF1, FGF2 or FGF7 for 1 or 24 hours. Immunohistochemistry was performed for E-cadherin and DAPI (FIG. 17A). Images were captured by fluorescence microscopy. Scale bars are as indicated. Immunohistochemistry was performed for β-catenin and DAPI (FIG. 17B). Images were captured by fluorescence microscopy. Scale bars are as indicated. Immunohistochemistry was performed for E-cadherin, phospho-β-catenin and DAPI (FIG. 17C). Images were captured by fluorescence microscopy. Scale bars are as indicated.

FIG. 18 illustrates that syndecan-1 increases survival in an inflammatory bowel disease mouse model. Wild-type and syndecan-1 knock-out (Sdc1 KO) mice were administered 10% DSS in their drinking water. Overall survival was measured over eight days (FIG. 18A). Weight of surviving mice was measured over seven days (FIG. 18B).

### DETAILED DESCRIPTION

Described herein are compositions of and methods for using proteins (e.g., FGF), proteoglycans (e.g., syndecans), agents that modulate proteoglycans and agents that modulate signaling (e.g., Wnt signaling) for the regulation of inflammatory responses and/or the treatment of diseases associated with an inflammatory response (e.g., diseases associated with an improper immune response). The therapeutics provided herein can be used to treat any disease associated with an inflammatory response. Such diseases include, for example, IBD.

IBD is one of several conditions where the pathology associated with the disease stems primarily from an excessive and unchecked inflammatory process. Approximately 1 in every 10,000 people is diagnosed with IBD each year. Although the incidence of IBD is relatively low, its prevalence is much greater as IBD is a chronic disease with no known root cause or cure. Patients currently only attempt to manage their disease with anti-inflammatory drugs often not designed specifically for IBD. Furthermore, the administration of these drugs is more often unpleasant for the patient, which reduces compliance and, therefore, treatment success. IBD is hypothesized to derive from an inappropriate and continuous activation of the mucosal immune system. The response may stem from defects in one or both of the intestinal epithelium and the mucosal immune systems [61]. Failure to maintain epithelial integrity, either as a primary epithelial defect or secondary to inflammation, can lead to increased antigen uptake and the associated colitis phenotype [62,63]. Epithelial repair is, therefore, important in the resolution of colonic inflammation and therapy for IBD [5].

Members of the FGF family have a protective effect in the colonic epithelium and can serve to support angiogenesis and promote bowel wall thickening in IBD. FGFs, such as FGF2, FGF7, FGF10 and FGF20, are normally derived from γδ T cells found in the colonic epithelium. These growth factors maintain the colonic epithelium and promote its recovery after insult by inducing the growth and migration of epithelial cells and can regulate the severity and progression of IBD [5, 13-15]. While various members of the FGF family have been demonstrated to have protective effects (e.g., by reducing inflammation) in cellular and animals models of IBD, this success has not been recapitulated in humans. For example, FGF2, FGF7 and FGF10 have been used in clinical trials for IBD with limited success. The FGF receptor 3b isoform is a predominant FGFR in the colonic epithelium, and although FGF2 and FGF7, which promote intestinal epithelial cell growth and wound healing [71], have been associated with FGFR3b, this receptor does not support their activity. Understanding the cellular mechanism by which FGFs can induce their protective and therapeutic responses in the colon can shed insight into their role in epithelial repair as well as in bowel wall thickening. Furthermore, mechanisms elucidated in the colon can provide insight into FGF activity in other physiological or pathological settings.

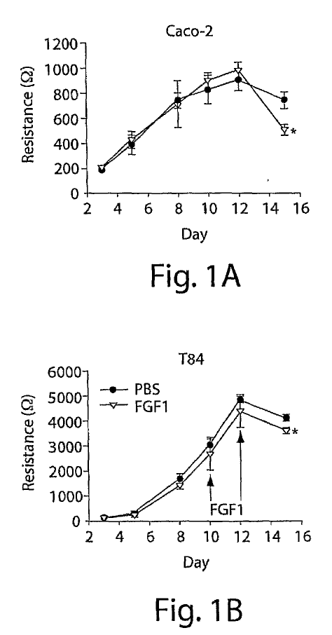

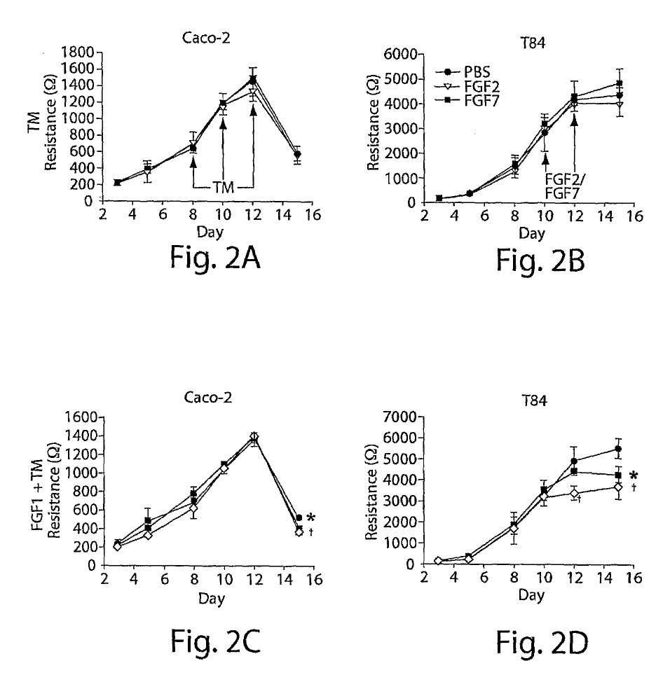

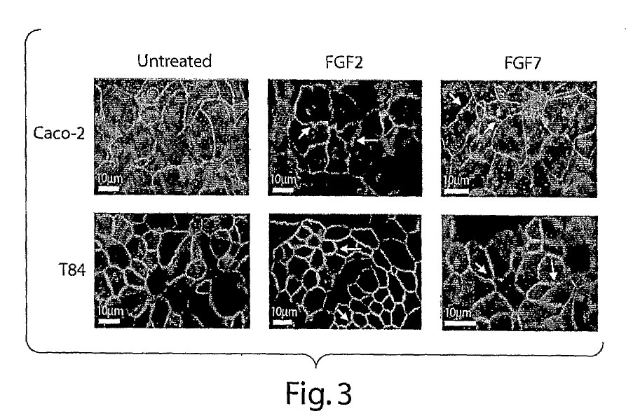

How various FGFs interact with each other and with epithelial cells to induce cellular mediated responses in the setting of IBD is presented herein. FGF2 and FGF7 promotion of cellular mediated responses in colonic epithelial cell lines was investigated. Using FGF2 and FGF7 as model FGFs, as well as the biochemical information available for these ligands, the effect of FGFs on the colonic epithelium was explored. Caco-2 and T84 cells predominantly expressed FGFR3b, through which FGF1 (but not FGF2 or FGF7) can induce a cellular response. FGF2 and FGF7, however, induced cellular responses that were dependent on FGFR3b and its downstream signaling. The activities of FGF2 and FGF7 were associated with increases in paracellular flux and alterations in the distribution of the tight junction proteins occludin and ZO-1. FGF1, secreted by both Caco-2 and T84 cells, was found to be necessary for FGF2 and FGF7 activity. FGF1, a factor derived from the epithelial cell itself, can reduce cell-cell adhesions, as measured by transepithelial resistance (TER) (FIG. 1). The reduction in TER occurs through the retraction of tight junction proteins including occludin and ZO-1 (FIG. 3). It is thought that FGF1 serves an important role in regulating FGF2 and FGF7 in the colon, and its expression by colonic epithelial cells enables FGF2 and FGF7 to promote epithelial maintenance and repair, while its downregulation leads to the angiogenesis and bowel wall thickening associated with FGF activity within the lamina propria in IBD.

Provided herein, therefore, are compositions and methods for altering an inflammatory response, treating a disease associated with an inflammatory response, etc., with FGF1 and at least one other FGF. As mentioned above, the FGF family consists of at least 23 members. All the members of the FGF family bind heparin and retain structural homology across species, suggesting a conservation of their structure/function relationship (Ornitz et al., J. Biol. Chem. 271(25):15292-15297, 1996.). A protein is a member of the FGF family, as used herein, if it shows significant sequence and three-dimensional structural homology to other members of the FGF family, FGF-like activity in in vitro or in vivo assays and binds to heparin or heparin-like substances. FGFs include, for example, FGF1, FGF2, FGF7, FGF10 and FGF20.

A preferred FGF for combination with FGF1, in some embodiments, is FGF2, FGF7, FGF10 or FGF20. In some embodiments, human FGFs are preferred for use in the compositions and methods of the invention. The terms “FGF1”, “FGF2”, “FGF7”, “FGF10” and “FGF20”, as used herein, refer to any FGF1, FGF2, FGF7, FGF10 and FGF20, respectively, that exhibits biologic activity. FGF1, therefore, includes but is not limited to the protein recognized as native FGF1 (See, e.g., GenBank Accession Number NM_{—}000800, NM_{—}033137, NM_{—}033136), truncated forms exhibiting activity, extended forms, higher molecular weight N-terminally extended forms, conservatively substituted versions of the native protein and functionally equivalent FGF1 derivatives of any of these. Likewise, FGF2 includes the protein recognized as native FGF2 (See, e.g., GenBank Accession Number NM_{—}002006), truncated forms, extended forms such as placental FGF, conservatively substituted versions of the native protein and functionally equivalent derivatives. FGF7 includes the protein recognized as native FGF7 (See, e.g., GenBank Accession Number NM_{—}002009), truncated forms, extended forms, conservatively substituted versions of the native protein and functionally equivalent derivatives. FGF10 includes the protein recognized as native FGF10 (See, e.g., GenBank Accession Number NM_{—}004465), truncated forms, extended forms, conservatively substituted versions of the native protein and functionally equivalent derivatives. Likewise, FGF20 includes the protein recognized as native FGF20 (See, e.g., GenBank Accession Number NM_{—}019851), truncated forms, extended forms, conservatively substituted versions of the native protein and functionally equivalent derivatives. The terms specifically include natural FGFs extracted from mammalian tissue as well as recombinant polypeptides expressed from DNA from any species. FGFs also include mutant versions of the wild-type FGF. In one embodiment, the mutant FGF is a mutant FGF2. The FGF2 mutant can be, for example, a heparin binding mutant (e.g., K26A, K119A/R120A or K125A) or a receptor binding mutant (Y103A, Y111A/W114A). Such mutants as well as a method for their production have been described in Padera et al., FASEB J. 13, 1677-1687 (1999).

As used herein, a “conservative amino acid substitution” or “conservative substitution” refers to an amino acid substitution in which the substituted amino acid residue is of similar charge as the replaced residue and is of similar or smaller size than the replaced residue. Conservative substitutions of amino acids include substitutions made amongst amino acids within the following groups: (a) the small non-polar amino acids, A, M, I, L, and V; (b) the small polar amino acids, G, S, T and C; (c) the amido amino acids, Q and N; (d) the aromatic amino acids, F, Y and W; (e) the basic amino acids, K, R and H; and (f) the acidic amino acids, E and D. Substitutions which are charge neutral and which replace a residue with a smaller residue may also be considered “conservative substitutions” even if the residues are in different groups (e.g., replacement of phenylalanine with the smaller isoleucine). The term “conservative amino acid substitution” also refers to the use of amino acid analogs or variants.

Methods for making amino acid substitutions, additions or deletions are well known in the art. The terms “conservative substitution”, “non-conservative substitutions”, “non-polar amino acids”, “polar amino acids”, and “acidic amino acids” are all used consistently with the prior art terminology. Each of these terms is well-known in the art and has been extensively described in numerous publications, including standard biochemistry text books, such as “Biochemistry” by Geoffrey Zubay, Addison-Wesley Publishing Co., 1986 edition, which describes conservative and non-conservative substitutions and properties of amino acids which lead to their definition as polar, non-polar or acidic.

The FGFs described herein can be in monomeric or multimeric (e.g., dimeric) form. An “FGF multimer” or “FGF in multimeric form” or “mFGF”, as used herein, refers to more than one FGF monomer liked to one another. Preferably, the FGF multimer contains an even number of FGF monomers. For example, the FGF multimer can be composed of two FGF dimers. The FGF dimers can be homodimers or they can be heterodimers, and the two FGF dimers of the FGF multimer can be identical or non-identical. For example, FGF multimers can include FGF1-FGF1/FGF2-FGF2, FGF1-FGF1/FGF7-FGF7, FGF2-FGF2/FGF7-FGF7, FGF1-FGF2/FGF1-FGF2, FGF1-FGF7/FGF1-FGF7, FGF2-FGF7/FGF2-FGF7, etc. As another example, the FGF multimer is a FGF dimer. An “FGF dimer” or “FGF in dimeric form” or “dFGF”, refers to two FGF monomers linked to one another. An FGF dimer can be a heterodimer or a homodimer. “Heterodimer” refers to two non-identical monomers linked together, while “homodimer” refers to two identical monomers linked together. Examples of FGF dimers, therefore, include FGF1-FGF2, FGF1-FGF7, FGF2-FGF7, FGF1-FGF1, FGF2-FGF2, FGF7-FGF7, etc. The multimers (e.g., dimers) can be composed of native FGFs or mutant FGFs or a combination thereof. In one embodiment, therefore, the FGFs of an FGF multimer (e.g., dimer) can include an FGF2 mutant as provided above (e.g., K26A, K119A/R120A, K125A, Y103A or Y111A/W114A). In another embodiment, all of the monomers of the FGF multimer (e.g., dimer) are a FGF2 mutant. The monomers can all be identical mutants or they can be non-identical mutants.

FGF dimers include modified FGF dimers and native FGF dimers that have been stabilized to maintain the dimeric state. A “modified FGF dimer”, as used herein, is an FGF dimer composed of two FGF monomers linked to one another, wherein the dimer includes at least one modification from a native FGF dimer. The modification may be within the amino acid sequence of one or both the FGF monomers or it may be the linkage itself. For instance, the modified FGF dimer may be composed of two naturally occurring FGF monomers which are linked by a linker molecule. A “stabilized FGF dimer” is one in which the monomers have a higher probability of remaining in a dimeric complex than monomeric FGF ordinarily would remain in a dimeric complex. The stabilized dimer may be accomplished through a variety of mechanisms. For example a linker molecule may be used to stabilize the dimeric structure of FGF. Covalent or other non-covalent interactions may also be used to stabilize the dimer, as long as the interactions form a more stable dimeric form of FGF than the non-covalent interactions between native FGF monomers. Examples of FGF dimers for use in the compositions and methods of the invention described herein as well as methods of their production are provided in copending U.S. application Ser. No. 10/108,195, the contents of which are incorporated herein by reference.

Compositions of and methods for altering an inflammatory response, treating a disease associated with an inflammatory response, etc., with an FGF multimer (e.g., dimer) are also provided. Such FGF multimers are provided as described above. Preferably, the FGF multimer is a FGF2 dimer. Even more preferably, the FGF dimer is a stabilized FGF2 dimer. Still more preferred is a stabilized FGF2 homodimer. In some embodiments where an FGF multimer is used to alter an inflammatory response, the inflammatory response is not associated with a wound, a scar, an ulcerating disease, inflammatory neuropathy or chronic demyelinating polyradiculoneuropathy. In some embodiments, when the method is one where a subject is administered an FGF multimer, the subject does not have a wound, a scar, an ulcerating disease, inflammatory neuropathy or chronic demyelinating polyradiculoneuropathy. In other embodiments, the FGF multimer is administered to a subject that does have a wound, a scar, an ulcerating disease, inflammatory neuropathy or chronic demyelinating polyradiculoneuropathy but also has cells affected by an inflammatory response not associated with the wound, scar, ulcerating disease, inflammatory neuropathy or chronic demyelinating polyradiculoneuropathy. In some embodiments, an FGF multimer is administered such that these cells only or preferentially are contacted with the FGF multimer. By preferentially, it is intended that more of the cells affected by an inflammatory response not associated with the wound, scar, ulcerating disease, inflammatory neuropathy or chronic demyelinating polyradiculoneuropathy are contacted with an FGF multimer than the cells associated with the wound, scar, ulcerating disease, inflammatory neuropathy or chronic demyelinating polyradiculoneuropathy. In some embodiments, 25%, 30%, 40%, 50%, 75%, 100%, 200%, 300%, 500% or more desired cells are contacted than the cells associated with the wound, scar, ulcerating disease, inflammatory neuropathy or chronic demyelinating polyradiculoneuropathy. Such administration can be accomplished with many ways known in the art and dependent on the cells that are intended to be contacted with the FGF multimer and those that are not. One of ordinary skill in the art will recognize that such administration can be accomplished with the use of targeting molecules or localized administration.

The mechanism by which FGF1 enabled FGF2 and FGF7 to promote a cellular response is also herein described. FGF1 was found to induce the localization of syndecan-1, which co-localized, with lipid rafts, to the nucleus. These results not only identify syndecan-1 as an important factor in enabling FGF2 and FGF7 activity, but also suggest that it has a protective effect in IBD. FGF2 and FGF7 activity is also associated with syndecan clustering. In addition, wild-type mice resist acute colitis much more effectively than sydecan-1 knock-out mice.

Compositions and methods for using proteoglycans, such as syndecans, are, therefore, also provided. As used herein, “proteoglycan” refers to molecules that contains both a protein and a glycosaminoglycan. Proteoglycans include HSPGs, chondroitin sulfate proteoglycans (CSPGs) and keratan sulfate proteoglycans (KSPGs). HSPGs include proteoglycans that contain heparan sulfate (HS). Examples of HSPGs include syndecans, glypicans, perlecans, etc. CSPGs include proteoglycans that contain chondroitin sulfates. Some examples of CSPGs include versican, decorin, neurocan, NG2, phosphacan and brevican. KSPGs include proteoglycans that contain keratan sulfates, such as lumican. The terms HSPGs, CSPGs and KSPGs are used herein consistent with their use in the relevant art. Cell surface HSPGs, such as syndecans, are essential modulators of physiological and pathological processes [34, 36-38]. Syndecans can regulate actin stress fiber formation [34], PSD95/DLG/ZO-1 (PDZ) protein activity including that of zona occludens-1 (ZO-1) [54,55,69,70] and Wnt-1 signaling [68].

The syndecan family of proteoglycans has been widely implicated in the maintenance of epithelial morphology and formation of focal contacts. These cell surface proteoglycans have a membrane bound core protein. The core protein of syndecans contains eight multifunctional domains. The ectodomain of the syndecan family has regions for glycosaminoglycan attachment, cell interaction, proteolytic cleavage and oligomerization (Bernfield M et al., 1999). This ectodomain of the syndecans is constitutively shed. Ectodomain shedding is a proteolytic mechanism of releasing the extracellular domains of cell surface proteins as soluble ectodomains. Ectodomain shedding is mediated by peptide hydroxamate-sensitive metalloproteinases, which are collectively called sheddases or secretases (J Schlondorff et al., 1999). Ectodomain shedding can also be affected by several external factors such as stress and activation of several signaling pathways (Bernfield M et al., 1999). By way of their HS chains, syndecans can bind a wide variety of soluble and insoluble extracellular ligands (Bernfield M et al., 1999). All adherent cells express at least one syndecan and some express multiple syndecans. Syndecan shedding is an important element in Wnt signaling [5]. Syndecan shedding occurs with proteolytic cleavage of the core protein within nine amino acids of the outer leaflet of the plasma membrane [29,128]. Glypicans can also be shed via glycosylphosphatidylinositol-specific phospholipases [86]. After shedding, the HSPG ectodomains retain their ligand binding properties. Shed syndecans can, for example, inhibit FGF2 activity [219].

Syndecans, as used herein, include syndecan-1 (See, e.g., GenBank Accession Number NM_{—}001006946, NM_{—}002997), syndecan-2 (See, e.g., GenBank Accession Number NM_{—}002998), syndecan-3 (See, e.g., GenBank Accession Number NM_{—}014654) and syndecan-4 (See, e.g., GenBank Accession Number NM_{—}002999). Syndecan-1, was first identified as a developmentally regulated type-I transmembrane protein that bound extracellular-matrix (ECM) components to epithelial cells. Proteoglycans (e.g., syndecans), therefore, can be present in a composition or used in a method as provided herein alone or in combination. In one embodiment, the complete proteoglyean (e.g., syndecan) is present in a composition or used in a method provided herein. In another embodiment, the ectodomain of a proteoglycan (e.g., syndecan) is present in a composition or used in a method provided herein. As used herein, “ectodomain” refers to the portion of a proteoglycan (e.g., syndecan) that is extracellular, including the attached glycosaminoglycan(s), or a fragment thereof that maintains a function of the extracellular portion. The term is, therefore, also intended to include functional equivalents of the extracellular portion.

Proteoglycans, such as syndecans, or their ectodomains can be obtained with any of a number of methods known in the art. As an example, cells that express the desired proteoglycan can be obtained and/or cultured in vitro and the ectodomains cleaved and purified with, for example, electrophoretic techniques. As another example, cells can be engineered to produce the proteoglycans (e.g., syndecans) or their ectodomains in vitro, and the resulting proteoglycans or ectodomains purified. An example of this can be found, for example, in Yang et al., Blood, Vol. 100, No. 2, 2002.

The compositions and methods provided can also include or include the use of, respectively, agents that cause the increased production of proteoglycans or their ectodomains. Such agents include nucleic acid molecules that encode the sequence of a proteoglycan or its ectodomain (e.g., a vector) or a host cell containing such a nucleic acid molecule. As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in a host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art, and genes which visibly affect the phenotype of transformed or transfected cells, hosts, colonies or plaques. Preferred vectors are those capable of autonomous replication and expression of the structural gene products present in the DNA segments to which they are operably joined.

As used herein, a coding sequence and regulatory sequences are said to be “operably joined” when they are covalently linked in such a way as to place the expression or transcription of the coding sequence under the influence or control of the regulatory sequences. As used herein, “operably joined” and “operably linked” are used interchangeably and should be construed to have the same meaning. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region is operably joined to a coding sequence if the promoter region is capable of effecting transcription of that DNA sequence such that the resulting transcript can be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Often, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

The compositions and methods provided can also include or include the use of, respectively, agents that modulate proteoglycans. As used herein, an agent that “modulates proteoglycans” refers to any agent that modulates the association of a proteoglycan to the cell surface and/or cleaves the proteoglycan to release the ectodomain. Such agents include agents that increase or promote proteoglycan shedding. Increased shedding may also be the result of increased production. As used herein, “shedding” refers to the release or cleavage of the ectodomain of a proteoglycan. Agents that modulate proteoglycans include, for example, IL-1β, IL-6, TNF-α, IL-1α, IL-4, IL-5, TGFβ-1, TGFβ-2, matrilysin, FGF2, EGF, PIF and RANTES. Such agents also include tetrathiomolybdate (TMB), protein kinase C (PKC) activators and inhibitors, PKC, phorbol 12-myristate 13-acetate (PMA), matrix metalloproteinases (MMPs) and tissue inhibitors of metalloproteinases (TIMPs). In some embodiments the agent is matrilysin, EGF or PIF. In some embodiments the agent is not tetrathiomolybdate.

As used herein, a “proteoglycan agent” is intended to refer to proteoglycans themselves as well as agents that increase proteoglycan production and/or shedding. Likewise, a “syndecan agent” refers to syndecans themselves as well as agents that increase syndecan production and/or shedding.

Compositions are, therefore, provided that include proteoglycan agents (e.g., syndecan agents) as are methods of their use. Such methods include methods for altering an inflammatory response as well as methods for treating a disease associated with an inflammatory response.

It has also been found that Wnt signaling was affected by FGF2 and FGF7, consistent with a syndecan-1-dependent mechanism. Wnt signaling can be altered in numerous ways, including antibodies to ligands and receptors, as well as the addition of specific ligands. Therefore, also provided herein are compositions that include, as well as methods for using, an agent that alters Wnt signaling. As used herein, an “agent that alters Wnt signaling” is any agent that upregulates or downregulates the Wnt signaling pathway or any portion thereof. Preferably, the agent alters the Wnt signaling pathway and alters an inflammatory response. Also preferred are agents that alter the Wnt signaling pathway and can treat a disease associated with an inflammatory response. Agents that alter Wnt signaling include agents that can activate or inhibit any of the proteins that are associated with the Wnt signaling pathway. The proteins associated with the Wnt signaling pathway are known in the art (See, e.g., Huelsken and Behrens, Journal of Cell Science, 115(21), 3977-3978, 2002). Agents that activate include, for example, Disheveled and inhibitors (e.g., antibodies) to Dikkopf, while agents that inhibit include, for example, Dikkopf, inhibitors (e.g., antibodies) to Frizzled and inhibitors (e.g., antibodies) to Disheveled. Agents that alter Wnt signaling include the proteins that are associated with Wnt signaling themselves, such as Wnt1, Wnt3a, Wnt5 and Wnt10.

Modulation (e.g., with antibodies) of beta-1 integrin, beta-3 integrin, alpha-2 integrin, alpha-4 integrin and alpha-5 integrin can also be used in the compositions and methods provided herein.

The compositions provided herein can be used to alter an inflammatory response (e.g., by altering FGF activity and/or directly inducing cellular changes associated with altering an inflammatory response). As used herein, “altering an inflammatory response” is meant to refer to any change to an inflammatory process, some portion of an inflammatory process or the effects thereof. “Altering” is also referred to herein as “modulating”. Such changes are intended to include an increase or decrease in inflammation or an increase or decrease in the effects of inflammation. Preferably, in some embodiments, the alteration results in a decrease in inflammation. The compositions provided can be used for the treatment of a subject who has a disease associated with an inflammatory response. In some embodiments, the compositions can be used to effect cell proliferation, wound healing and/or protection against an inflammatory response. Therefore, the compositions provided can be used in the treatment of a subject that has a disease associated with an inflammatory response. The agents provided herein, in some embodiments, can be contacted with cells affected by an inflammatory response. As used herein, “cells affected by an inflammatory response” are those that are directly involved in an inflammatory process and/or are subjected to the results of an inflammatory process. These cells, therefore, can be those that are in an area where there is inflammation.

The compositions and methods provided can be used to treat any disease that is associated with an inflammatory response, which includes diseases that are associated with an improper immune response. A “disease that is associated with an improper immune response” is any disease in which an inflammatory response occurs and such an inflammatory response is undesirable and/or occurs at levels that are undesirable. The disease can, therefore, be one where there is unwanted inflammation. Diseases that are associated with an improper immune response include inflammatory diseases, autoimmune diseases (e.g. lupus), chronic disease with bouts of acute inflammation, Lyme disease, tuberculosis, exuberant granulomas, keloids and multiple myeloma. Furthermore, the compositions and methods provided herein can also be used in promoting cell proliferation, angiogenesis, wound healing, treatment of ulcers (e.g., diabetic ulcers) and scar reduction after surgery. As used herein, “disease” is intended to include disorders and conditions.

The terms “treat” and “treating”, as used herein, refer to inhibiting completely or partially an undesirable biological effect of a disease, as well as inhibiting any increase in an unwanted biological effect of a disease. The terms “treat” or “treating” are also intended to include inhibiting completely or partially an inflammatory response and/or resulting inflammation. The terms are further intended to include promoting a desirable biological effect to provide some benefit to a subject with a disease as provided herein.

The subject that can be treated with a composition provided here can have an inflammatory disease. In some embodiments the inflammatory disease is non-autoimmune inflammatory bowel disease, post-surgical adhesions, coronary artery disease, hepatic fibrosis, acute respiratory distress syndrome, acute inflammatory pancreatitis, endoscopic retrograde cholangiopancreatography-induced pancreatitis, burns, atherogenesis of coronary, cerebral and peripheral arteries, appendicitis, cholecystitis, diverticulitis, visceral fibrotic disorders, wound healing, skin scarring disorders (keloids, hidradenitis suppurativa), granulomatous disorders (sarcoidosis, primary biliary cirrhosis), asthma, pyoderma gandrenosum, Sweet's syndrome, Behcet's disease, primary sclerosing cholangitis or an abscess. In some preferred embodiment the inflammatory disease is inflammatory bowel disease (e.g., Crohn's disease or ulcerative colitis).

The inflammatory disease can be an autoimmune disease. The autoimmune disease in some embodiments is rheumatoid arthritis, rheumatic fever, ulcerative colitis, Crohn's disease, autoimmune inflammatory bowel disease, insulin-dependent diabetes mellitus, diabetes mellitus, juvenile diabetes, spontaneous autoimmune diabetes, gastritis, autoimmune atrophic gastritis, autoimmune hepatitis, thyroiditis, Hashimoto's thyroiditis, insulitis, oophoritis, orchitis, uveitis, phacogenic uveitis, multiple sclerosis, myasthenia gravis, primary myxoedema, thyrotoxicosis, pernicious anemia, autoimmune haemolytic anemia, Addison's disease, Anklosing spondylitis, sarcoidosis, scleroderma, Goodpasture's syndrome, Guillain-Barre syndrome, Graves' disease, glomerulonephritis, psoriasis, pemphigus vulgaris, pemphigoid, excema, bulous pemiphigous, sympathetic opthalmia, idiopathic thrombocylopenic purpura, idiopathic feucopenia, Sjogren's syndrome, systemic sclerosis, Wegener's granulomatosis, poly/dermatomyositis, primary biliary cirrhosis, primary sclerosing cholangitis, lupus or systemic lupus erythematosus.

The subject can, in some embodiments, be in need of wound healing or scar reduction. As used herein, a subject that is “in need of wound healing or scar reduction” is a subject with a wound, scar or some sort of tissue injury (e.g., tissue injured as a result of an inflammatory process) in which the therapeutics provided herein would have some benefit.

Furthermore, the subject can be one with an ulcerating disease. Ulcerating diseases include regional ileitis of the gastrointestinal tract, ulcerative colitis and peptic ulcers (either duodenal or gastric).

The subject can also be one with Lyme disease, tuberculosis, exuberant granulomas, keloids, multiple myeloma or an ulcer (e.g., diabetic ulcer).

Each of the conditions, diseases or disorders recited herein is well-known in the art and/or is described, for instance, in Harrison's Principles of Internal Medicine (McGraw Hill, Inc., New York), which is incorporated by reference.

The compositions provided can include an additional therapeutic agent. Similarly, the methods provided can also include contacting or administering an additional therapeutic agent. An “additional therapeutic agent” is any agent that can result is some benefit for any disease, or provide some benefit to any subject, provided herein with the compositions of the invention and that is in addition to the compositions of the invention.

The additional therapeutic agent can be an anti-inflammatory agent. Anti-inflammatory agents include Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflunidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids and Zomepirac Sodium.

The additional therapeutic agent can be an agent for treating IBD. Agents for treating IBD include sulfasalazine, corticosteroids, immunosuppressives, nonsteroidal anti-inflammatory drugs (NSAIDs) and aminosalicylates (e.g., 5-ASA agents).

The additional therapeutic agent can be an anti-ulcer agent. Anti-ulcer agents include H2-blockers, acid pump inhibitors, mucosal protective medications, agents for treating reflux, 5-ASA and sulfasalazine.

The additional therapeutic agent can be an anti-cancer agent. Anti-cancer agents include Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin and Zorubicin Hydrochloride.

The additional therapeutic agent can be an anti-diabetic agent. Anti-diabetic agents include Avandamet, Fortamet, Metaglip, glucosidase inhibitors, insulin such as Humalog® (insulin lispro) Humulin, Novolin, Lantus, Apidra, a Meglitinide (e.g., Prandin or Starlix), sulfonylureas, a Thiazolidinedione (e.g., Actos® (pioglitazone hydrochloride)), Avandamet® (rosiglitazone maleate and metformin hydrochloride) and Avandia® (rosiglitazone maleate).

It has also been found that FGFs can regulate cell junctions. Therefore, compositions comprising FGFs can also be used to modulate intercellular junctions. The compositions can also be used to enhance the delivery of a therapeutic agent. Methods for modulating intercellular junction and/or enhancing the delivery of an additional therapeutic agent are also provided.

As used herein to “transiently disrupt” means to temporarily disturb one or more intercellular junctions such that the junction(s) no longer links the cells and/or are more permeable than in the native state or in the presence of an intercellular junction opening inhibitor (i.e., any agent that can inhibit intercellular junction disruption and/or decrease intercellular permeability). As used herein “intercellular junction” includes the points where cells are joined and/or the areas of the cells in between which the passage of molecules is inhibited or retarded as compared to the native state or to the passage of the molecules in the presence of an intercellular junction opening inhibitor. Intercellular junctions include tight junctions, adherens junctions, desmosomes, etc. In some instances in the literature “intracellular” has been used instead of “intercellular”.

The methods provided include methods for enhancing the delivery of one or more therapeutic agents to target tissues by transiently disrupting the intercellular seals formed by the tight junctions. As used herein “enhanced” means to increase the delivery of the therapeutic agent to a particular cell or tissue or to systemic circulation generally. The term also refers to the increased passage of a therapeutic agent via paracellular transport. In some embodiments the enhanced delivery refers to the passage of a therapeutic agent that otherwise would not be able to traverse the intercellular junctions. In other embodiments the delivery is enhanced 5%, 10%, 15%, 20%, 25%, 30%, 40%, 50%, 60%, 70%, 80%, 90%, 100%, 200%, 300%, 500%, 1000% or more than the delivery that would be accomplished without the use of a FGF composition as provided herein.

The therapeutic agent can be administered prior to, concurrently with or after the FGF composition is administered. Preferably, the therapeutic agent and FGF composition are administered so that the delivery of the therapeutic agent is enhanced because of the disruption of intercellular junctions. In some embodiments the FGF composition is administered 1, 2, 3, 4, 5, 10, 15, 20, 25, 30, 60 or more minutes prior to the administration of the therapeutic agent. In other embodiments the FGF composition is administered 1, 2, 3, 4, 5, 6, 7, 8, 9, 10, 15, 20 or more hours prior to the administration of the therapeutic agent. In still other embodiments the FGF composition is administered at least 10 minutes prior to the administration of the therapeutic agent. In still other embodiments the FGF composition is administered no more than 1, 2, 3, 4, 5, 6, 7, 8, 9, or 10 hours before the administration of the therapeutic agent. In still other embodiments the FGF composition is administered anywhere from 1 minute to 10 hours prior to the administration of the therapeutic agent.

In some embodiments the therapeutic agent is administered 1, 2, 3, 4, 5, 10, 15, 20, 25, 30, 60 or more minutes prior to the administration of the FGF composition. In other embodiments the therapeutic agent is administered 1, 2, 3, 4, 5, 6, 7, 8, 9, 10, 15, 20, 30, 40, 50, 60, 75, 100 or more hours prior to the administration of the FGF composition In still other embodiments the therapeutic agent is administered at least 10 minutes prior to the administration of the FGF composition. In yet other embodiments the therapeutic agent is administered 1, 2, 3, 4, 5, 10, 20, 30 or more days prior to the administration of the FGF composition. In further embodiments the therapeutic agent is administered 1, 2, 3, 4, 5, 10, 20, 30 or more months prior to the administration of the FGF composition. In yet other embodiments the therapeutic agent is administered 1, 2, 3, 4, 5 or more years prior to the administration of the FGF composition. In some embodiments, therefore, administration can be accomplished with the use of slow release materials, such as polyanhydride wafers or block copolymers, such as poly(ethylene glycol) (PEG)/poly(lactic-co-glycolic acid) (PLGA) block copolymers as an example. In still other embodiments the therapeutic agent is administered no more than 1, 2, 3, 4, 5, 6, 7, 8, 9, 10, 15, 24, 36 or 72 hours before the administration of the FGF composition.

The FGF and the therapeutic agent can be linked in some embodiments. In other embodiments the FGF and/or the therapeutic agent can be linked to a targeting molecule. Methods of linking compounds as well as linkers are well known in the art.

Effective amounts (or amounts effective) of the compositions of the invention are administered to subjects in need of such treatment. Effective amounts are those amounts which will result in some benefit to the subject (e.g., a desired improvement in the disease or symptoms associated therewith). Such amounts can be determined with no more than routine experimentation. As used herein, an amount “effective to alter an inflammatory response” is any amount of the agents of the invention alone or in combination with an additional therapeutic agent to change (e.g., inhibit) an inflammatory process or portion thereof and/or reduce or eliminate an outcome of the inflammatory process. Preferably, in some embodiments the amount effective can be one that leads to a reduction or elimination of one or more effects of inflammation. In some embodiments, the amount effective is one in which there is some protective effect and/or promotion of proliferation or wound healing. In still other embodiments the effective amount is one in which proteoglycan shedding is increased.

In other embodiments, FGF compositions are in effective amounts to transiently disrupt and/or enhance the delivery of a therapeutic agent (e.g., through a cell barrier).

It is believed that doses ranging from 1 nanogram/kilogram to 100 milligrams/kilogram, depending upon the mode of administration, will be effective. In some embodiments the level of administration is between 5 nanograms to 14 milligrams per 4 square centimeter area of cells. In other embodiments the level of administration is 100 ng/ml per square centimeter area of cells. In still other embodiments the level of administration is 50 ng/ml per square centimeter area of cells. The absolute amount will depend upon a variety of factors (including whether the administration is in conjunction with other methods of treatment, the number of doses and individual patient parameters including age, physical condition, size and weight) and can be determined with routine experimentation. It is preferred, generally, that a maximum dose be used, that is, the highest safe dose according to sound medical judgment. The mode of administration may be any medically acceptable mode including oral, ocular, topical, transdermal, rectal, nasal, subcutaneous, intravenous, etc. or via administration to a mucous membrane. In some embodiments the mode of administration is topical administration. Preferably, in some embodiments, the mode of administration is oral, intravenous or by enema/per rectum.

In general, when administered for therapeutic purposes, the formulations of the invention are applied in pharmaceutically acceptable solutions. Such solutions can be sterile. Such preparations may routinely contain pharmaceutically acceptable concentrations of salt, buffering agents, preservatives, compatible carriers, adjuvants and optionally other therapeutic ingredients.

The compositions of the invention may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, pharmaceutically acceptable salts can be prepared as alkaline metal or alkaline earth salts, such as sodium, potassium or calcium salts of the carboxylic acid group.

Suitable buffering agents include: acetic acid and a salt (1-2% W/V); citric acid and a salt (1-3% W/V); boric acid and a salt (0.5-2.5% W/V); and phosphoric acid and a salt (0.8-2% W/V). Suitable preservatives include benzalkonium chloride (0.003-0.03% W/V); chlorobutanol (0.3-0.9% W/V); parabens (0.01-0.25% W/V) and thimerosal (0.004-0.02% W/V).

The present invention provides pharmaceutical compositions, for medical use, which comprise the one or more agents of the invention together with one or more pharmaceutically acceptable carriers and optionally other therapeutic ingredients. The pharmaceutical compositions can also, in some embodiments, include one or more additional therapeutic agents. The term “pharmaceutically-acceptable carrier” as used herein, and described more fully below, means one or more compatible solid or liquid filler, dilutants or encapsulating substances which are suitable for administration to a human or other animal. In the present invention, the term “carrier” denotes an organic or inorganic ingredient, natural or synthetic, with which the active ingredient is combined to facilitate the application. The components of the pharmaceutical compositions also are capable of being commingled with each other, in a manner such that there is no interaction which would substantially impair the desired pharmaceutical efficiency. Pharmaceutically acceptable carriers can, in some embodiments, be sterile.

The compositions will be provided in different vessels, vehicles or formulations depending upon the disorder and mode of administration. For example, and as described in greater detail herein, for oral application, the compounds can be administered as sublingual tablets, gums, mouth washes, toothpaste, candy, gels, films, etc.; for ocular application, as eye drops in eye droppers, eye ointments, eye gels, eye packs, as a coating on a contact lens or an intraocular lens, in contacts lens storage or cleansing solutions, etc.; for topical application, as lotions, ointments, gels, creams, sprays, tissues, swabs, wipes, etc.; for vaginal or rectal application, as an ointment, a tampon, a suppository, a mucoadhesive formulation, etc.

A variety of other administration routes are also available. The particular mode selected will depend, of course, upon the particular active agent(s) selected, the desired results, the particular condition being treated and the dosage required for therapeutic efficacy. The methods of this invention, generally speaking, may be practiced using any mode of administration that is medically acceptable, meaning any mode that produces effective levels of inflammatory response alteration without causing clinically unacceptable adverse effects. One mode of administration is the parenteral route. The term “parenteral” includes subcutaneous injections, intravenous, intramuscular, intraperitoneal, intrasternal injection or infusion techniques. Other modes of administration include oral, mucosal, rectal, vaginal, sublingual, intranasal, intratracheal, inhalation, ocular, transdermal, etc. In some embodiments the administration of the compositions does not occur via the pulmonary route.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally, the oral formulations may also be formulated in saline or buffers for neutralizing internal acid conditions or may be administered without any carriers.

One suitable oral form is a sublingual tablet. A sublingual tablet delivers the composition to the sublingual mucosa. As used herein, “tablet” refers to pharmaceutical dosage forms prepared by compressing or molding. Sublingual tablets are small and flat, for placement under the tongue and designed for rapid, almost instantaneous disintegration and release the composition to the sublingual mucosa. The term “disintegration” means breaking apart. Preferably, the sublingual tablets of the present invention disintegrate, to release the composition, within five minutes and, more preferably, within a two minute period of time. Oral formulations can also be in liquid form. The liquid can be administered as a spray or drops to the entire oral cavity including to select regions such as the sublingual area. The sprays and drops of the present invention can be administered by means of standard spray bottles or dropper bottles adapted for oral or sublingual administration. The liquid formulation is preferably held in a spray bottle, fine nebulizer, or aerosol mist container, for ease of administration to the oral cavity. Liquid formulations may be held in a dropper or spray bottle calibrated to deliver a predetermined amount of the composition to the oral cavity. Bottles with calibrated sprays or droppers are known in the art. Such formulations can also be used in nasal administration.

The compositions of the invention can also be formulated as oral gels. As an example, the composition may be administered in a mucosally adherent, non-water soluble gel. The gel is made from at least one water-insoluble alkyl cellulose or hydroxyalkyl cellulose, a volatile nonaqueous solvent, and the composition. Although a bioadhesive polymer may be added, it is not essential. Once the gel is contacted to a mucosal surface, it forms an adhesive film due primarily to the evaporation of the volatile or non-aqueous solvent. The ability of the gel to remain at a mucosal surface is related to its filmy consistency and the presence of non-soluble components. The gel can be applied to the mucosal surface by spraying, dipping, or direct application by finger or swab.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Medical devices for the inhalation of therapeutics are known in the art. In some embodiments the medical device is an inhaler. In other embodiments the medical device is a metered dose inhaler, diskhaler, Turbuhaler, discus or a spacer. In certain of these embodiments the inhaler is a Spinhaler (Rhone-Poulenc Rorer, West Malling, Kent). Other medical devices are known in the art and include the following technologies Inhale/Pfizer, Mannkind/Glaxo and Advanced Inhalation Research/Alkermes.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. In some embodiments the compounds provided are administered by infusion pump. In some of these embodiments the compounds are administered by infusion pump. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990 and Langer and Tirrell, Nature, 2004 Apr. 1; 428(6982): 487-92, which are incorporated herein by reference.

The compositions may conveniently be presented in unit dosage form and may be prepared by any of the methods well known in the art of pharmacy.

In some embodiments the composition that is administered is in powder or particulate form rather than as a solution. Examples of particulate forms contemplated as part of the invention in some embodiments are provided in U.S. patent application Ser. No. 09/982,548, filed Oct. 18, 2001, which is hereby incorporated by reference in its entirety. In other embodiments the compositions are administered in aerosol form. In other embodiments the method of administration includes the use of a bandage, slow release patch, engineered or biodegradable scaffold, slow release polymer, tablet or capsule.

Other delivery systems can include time-release, delayed release or sustained release delivery systems. Such systems can avoid repeated administrations of the compounds of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. Specific examples include, but are not limited to: (a) erosional systems in which the agent is contained in a form within a matrix, found in U.S. Pat. Nos. 4,452,775 (Kent); 4,667,014 (Nestor et al.); and 4,748,034 and 5,239,660 (Leonard) and (b) diffusional systems in which an agent permeates at a controlled rate through a polymer, found in U.S. Pat. Nos. 3,832,253 (Higuchi et al.) and 3,854,480 (Zaffaroni). In addition, a pump-based hardware delivery system can be used, some of which are adapted for implantation.

Controlled release can also be achieved with appropriate excipient materials that are biocompatible and biodegradable. These polymeric materials which effect slow release may be any suitable polymeric material for generating particles, including, but not limited to, nonbioerodable/non-biodegradable and bioerodable/biodegradable polymers. Such polymers have been described in great detail in the prior art. They include, but are not limited to: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, hyaluronic acid, and chondroitin sulfate. In one embodiment the slow release polymer is a block copolymer, such as poly(ethylene glycol) (PEG)/poly(lactic-co-glycolic acid) (PLGA) block copolymer.

Examples of preferred non-biodegradable polymers include ethylene vinyl acetate, poly(meth) acrylic acid, polyamides, copolymers and mixtures thereof.

Examples of preferred biodegradable polymers include synthetic polymers such as polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. The most preferred polymers are polyesters, polyanhydrides, polystyrenes and blends thereof.

In another embodiment slow release is accomplished with the use of polyanhydride wafers.

The compositions can be administered locally or the compositions can further include a targeting molecule. The targeting molecule can be attached to an agent and/or an additional therapeutic agent or some combination thereof. A targeting molecule is any molecule or compound which is specific for a particular cell or tissue and which can be used to direct the agents provided herein to a particular cell or tissue. The targeted molecules can be any molecule that is differentially present on a particular cell or in a particular tissue. These molecules can be proteins expressed on the cell surface.

Targeting molecules can in some embodiments be used to target disease markers. For instance, the targeting molecule may be a protein (e.g., an antibody) or other type of molecule that recognizes and specifically interacts with a disease antigen.

Some aspects of the invention also encompass kits. The kits of the invention include one or more of the agents of the invention. The kits can further include one or more additional therapeutic agents, administration devices and/or instructions for use. The kits provided can also include a detection system. Detection systems can be used to determine the amount of any or all of the agents administered in the blood. Detection systems can be invasive or non-invasive. An example of an invasive detection system is one which involves the removal of a blood sample and can further involve an assay such as an enzymatic assay or a binding assay to detect levels in the blood. A non-invasive type of detection system is one which can detect the levels of the agent in the blood without having to break the skin barrier. These types of non-invasive systems include, for instance, a monitor which can be placed on the surface of the skin, e.g., in the form of a ring or patch, and which can detect the level of circulating agents. One method for detection may be based on the presence of fluorescence in the agent which is administered. Thus, if a fluorescently labeled agent is administered and the detection system is non-invasive, it can be a system which detects fluorescence. This is particularly useful in the situation when the patient is self-administering and needs to know the blood concentration or an estimate thereof in order to avoid side effects or to determine when another dose is required.

A subject is any human or non-human vertebrate, e.g., dog, cat, horse, cow, monkey, pig, mouse, rat.

The present invention is further illustrated by the following Examples, which in no way should be construed as further limiting. The entire contents of all the references (including literature references, issued patents, published patent applications, and co-pending patent applications) cited throughout this application are herein incorporated by reference.

### EXAMPLES

### Example 1

### Fibroblast Growth Factor (FGF) 1 is Important for FGF2 and FGF7 Regulation of Epithelial Monolayer Integrity

### Materials and Methods

### Proteins and Reagents

Recombinant human FGF1 was from Amgen (Thousand Oaks, Calif.). Recombinant human FGF2 was produced as described [1]. Recombinant human FGF7 was from Sigma (St. Louis, Mo.). Mouse recombinant collagen IV was from BD Biosciences (Bedford, Mass.). LY294002, PD98059 and SB203580 were from Promega (Madison, Wis.). Rabbit polyclonal anti-human FGF1, rabbit polyclonal anti-human FGFR1 and rabbit polyclonal anti-human FGFR3 were from Santa Cruz Biotechnology (Santa Cruz, Calif.). FITC conjugated goat anti-rabbit was from Molecular Probes (Eugene, Oreg.). Caco-2 and T84 cells were from American Type Culture Collection (Manassas, Va.). Fetal bovine serum (FBS) was from Hyclone (Logan, Utah). Minimal essential medium (MEM), Dulbecco's modified Eagle medium (DMEM), Ham's F12 medium, McCoy's 5a medium, Hank's buffered saline solution (HBSS), phosphate buffered saline (PBS), RPMI-1640, L-glutamine, penicillin/streptomycin, sodium pyruvate, HEPES, sodium bicarbonate and non-essential amino acid solution were obtained from GibcoBRL (Gaithersburg, Md.). BaF3 cells transfected with various FGFRs were generously provided by Dr. David Ornitz (Washington University, St. Louis, Mo.).

### Cell Culture

Caco-2 cells were maintained in MEM supplemented with 10% FBS and 15 mM HEPES. T84 cells were maintained in a 1:1 mixture of DMEM and F12 medium supplemented with 5% FBS, 1.2 g/L sodium bicarbonate, 2.5 mM L-glutamine, 15 mM HEPES and 0.5 mM sodium pyruvate. All media was additionally supplemented with 100 mg/L penicillin and 100,000 U/L streptomycin. All cells were grown in 175 cm² flasks at 37° C. in a 5% CO₂ humidified incubator. Caco-2 cells were passaged twice a week at confluence. T84 cells were passaged once a week at confluence.

BaF3 cells transfected with various FGFRs were maintained as independent suspension cultures in propagation media composed of RPMI-1640 supplemented with 10% FBS, 100 μg/ml penicillin, 100 U/ml streptomycin, 1.5 mM L-glutamine and 1 ng/ml mouse recombinant IL-3. Cultures were grown in 75 cm² flasks at 37° C. in a 5% CO₂ humidified incubator and were passaged 1:10 by dilution three times a week.

### RT-PCR

Five μg of total RNA was isolated from each of Caco-2 and T84 cells using Trizol reagent (Life Tech, Rockville, Md.) followed by reverse transcription with random hexamers. Specific oligomers were designed based on published sequences in order to detect the expression of specific FGFR isoforms. Sequences of primer pairs corresponding to distinct FGFR isoforms were as follows: FGFR1b: 5′-TGG AGC AAG TGC CTC CTC-3′ (SEQ ID NO: 1) and 5′-ATA TTA CCA CTT CGA TTG GTC-3′ (SEQ ID NO: 2); FGFR1c: 5′-TGG AGC TGG AAG TGC CTC CTC-3′ (SEQ ID NO: 3) and 5′-GTG ATG GGA GAG TCC GAT AGA-3′ (SEQ ID NO: 4); FGFR2b: 5′-GTC AGC TGG GGT CGT TTC ATC-3′ (SEQ ID NO: 5) and 5′-CTG GTT GGC CTG CCC TAT ATA-3′ (SEQ ID NO: 6); FGFR2c: 5′-GTC AGC TGG GGT CGT TTC ATC-3′ (SEQ ID NO: 7) and 5′-GTG AAA GGA TAT CCC AAT AGA-3′ (SEQ ID NO: 8); FGFR3b: 5′ GTA GTC CCG GCC TGC GTG CTA-3′ (SEQ ID NO: 9) and 5′-GAC CGG TTA CAC AGC CTC GCC-3′ (SEQ ID NO: 10); FGFR3c: 5′-GTA GTC CCG GCC TGC GTG CTA-3′ (SEQ ID NO: 11) and 5′-TCC TTG CAC AAT GTC ACC TTT-3′ (SEQ ID NO: 12); and FGFR4: 5′-CCC TGC CGG GAT CGT GAC CCG-3′ (SEQ ID NO: 13) and 5′-TCG AAG CCG CGG CTG CCA AAG-3′ (SEQ ID NO: 14). To control for total cell protein, RT-PCR was also performed on β-actin using the primers 5′-GCC AGC TCA CCA TGG ATG ATG ATA T-3′ (SEQ ID NO: 15) and 5′-GCT TGC TGA TCC ACA TCT GCT GGA A-3′ (SEQ ID NO: 16). PCR was performed using the Advantage-GC cDNA kit from Clontech as per manufacturer's instructions (Palo Alto, Calif.). Prior to experimental use, primers were confirmed to detect and have specificity towards given FGFR isoforms using BaF3 cells transfected with various FGFRs.

### FACS Analysis

Caco-2 was grown close to confluence, and T84 cells were grown to confluence. Cells were washed with 20 ml PBS and treated with 3.5 ml trypsin-EDTA per 175 cm² flask for 20-60 minutes at 37° C., until cells detached. The cells were then resuspended in 6.5 ml medium and pelletted. Cells were resuspended in PBS supplemented with 5% bovine serum albumin (BSA). Cell suspensions were filtered to remove clumps and resuspended to a concentration of 5×10⁶ cells/ml based on the readings of an electronic cell counter. Aliquots of 100 μl cell suspension were kept on ice for the duration of the experiment. The primary antibody, rabbit anti-human FGFR3, was added to cells at a 1:500 dilution and incubated for 1 hour. After washing with PBS supplemented with BSA, the secondary antibody, goat anti-rabbit conjugated to FITC was added at a 1:250 dilution and incubated 30 minutes. Cells were washed with PBS supplemented with BSA, and FACS was performed using a Coulter Epics XL (Beckman Coulter, Miami, Fla.). Controls of no antibody, no primary antibody, and no secondary antibody were additionally performed.

### Transepithelial Resistance

Caco-2 cells were grown close to confluence, and T84 cells were grown to confluence. The apical chambers of transwell plates with 6.5 mm diameter and 3 μm pore size filters were pretreated with 50 μl of 45 μg/ml collagen IV in 60% ethanol 24 hours prior to plating and incubated overnight at room temperature. After reaching confluence, cells were washed with 20 ml PBS and treated with 3.5 ml trypsin-EDTA per 175 cm² flask for 20-60 min at 37° C. until cells detached. Cells were resuspended in 16.5 ml media appropriate to the cell type. Each apical chamber was supplemented with 82 μl cell solution. The basolateral chamber was filled with 1 ml propagation media. Filters were fed with new media every two days over the course of experiments.

Caco-2 and T84 cells were allowed to grow on transwells to form monolayers. Monolayer formation was examined every other day by light microscopy as well as by transepithelial resistance (TER) measurements using an EVOM ohmmeter (World Precision Instruments, Sarasota, Fla.). After monolayer formation, cells were treated with 100 ng/ml FGF1, 100 ng/ml FGF2, 100 ng/ml FGF7 or an equivalent volume (10 μl) of PBS. TER measurements were made either after 72 hours or at each of the 30 minutes, 1 hour, 2 hours, 4 hours, 9 hours, 24 hours and 48 hours timepoints. To determine the effects of multiple additions of ligand, FGFs or PBS were added initially and again after 2 days, and TER measurements were made after 0, 2 and 5 days.

To probe the mechanism by which various FGFs induced cell mediated effects, various media treatments were employed. Ammonium tetrathiomolybdate (TM) was added 24 hours prior to the addition of ligands to yield a final concentration of 200 μM, sufficient to prevent the release of FGF1 [3]. Similarly, sodium chlorate, which reduces cell HSPGs and the sulfation of associated HSGAGs, was added 24 hours prior to the addition of ligands to yield a final concentration of 50 mM [33,34]. Antibodies and kinase inhibitors were added immediately prior to ligands. Antibodies to FGF1, FGFR1 and FGFR3 were added at a 1:100 dilution. Kinase inhibitors were added to yield final concentrations of 50 μM LY294002, 20 μM PD98059, 1 μM SB203580. All experiments were performed in triplicate.

### BaF3 Proliferation Assay

The capacity for FGF2 and FGF7 to promote proliferative responses was performed as previously described with slight modification [35,36]. BaF3 cells expressing FGFR1c or FGFR3c, previously verified by RT-PCR [37], were washed and resuspended to a density of 2×10⁵ cells/ml in IL-3 deficient RPMI-1640. The cell suspension was added to each well of a 24 well plate at 1 ml/well. Each well was supplemented with FGF2 or FGF7 between 1 and 100 ng/ml. After a three day incubation at 37° C., 5% CO₂, proliferation was measured with an electronic cell counter. For antibody inhibition assays, antibodies to FGFR1 as well as those to FGFR3 were administered 1:100 just prior to the addition of ligand. FGFR1 and FGFR3 antibodies were also added to cells in the absence of ligand.

### Caco-2 and T84 Proliferation Assays

Caco-2 cells were grown to just prior to confluence, and T84 cells were grown to confluence. Each flask was washed with 20 ml PBS and treated with 3 ml trypsin-EDTA at 37° C. for 20-60 minutes until cells detached. Cells were centrifuged for 3 minutes at 195×g. The supernatant was aspirated, and the cells were resuspended in 10 ml media. The cell density was measured using an electronic cell counter, and the suspension was diluted to 50,000 cells/ml. The suspension was plated at 1 ml/well into 12-well tissue culture plates. After a 24 hour incubation in a 5% CO₂, 37° C. humidified incubator, the media was aspirated, the wells were washed with serum free media, and the cells were supplemented with media containing 0.1% FBS. The wells were then supplemented with 10 μl PBS, 50 mM sodium chlorate, or 200 μM TM, and incubated for 24 hours. Subsequently, cells were supplemented with 100 ng/ml FGF1, FGF2, FGF7, or 10 μl PBS, and incubated for 72 hours. Wells were then washed twice with PBS and treated with 0.5 ml trypsin-EDTA/well and incubated at 37° C. until cells detached. Whole cell number was determined using an electronic cell counter. Data was averaged over three experiments, each consisting of four wells per condition.

### Monolayer Flux

Cells were plated on transwells as described. Media from the basolateral chamber was removed every other day and replaced with new media. Resistances were followed for 10 days, and either 100 ng/ml FGF2, 100 ng/ml FGF7 or 10 μl PBS were added on days 10 and 12. On day 15, media was removed from both the apical and basolateral chambers. The basolateral chamber was filled with 1 ml HBSS. FITC conjugated to 3 kDa dextran was added to the apical chamber in a volume of 100 μl, at 1 mg/ml in HBSS. After 30, 60 and 90 minutes, 50 μl was removed from the basolateral chamber and transferred to a 96 well plate, and 50 μl HBSS was added to the basolateral chamber. Subsequently, the flux was determined by measurement of FITC-conjugated dextran that crossed the monolayer by measurement with a fluorimeter, stimulating at 480 nm and measuring emission at 530 nm.

### Immunohistochemistry

To determine FGFR and FGF1 expression, Caco-2 cells were grown to just prior to confluence, and T84 cells were grown to confluence on collagen-coated glass slides. Cells were washed with PBS supplemented with 5% BSA. To stain for FGFR3, rabbit anti-human FGFR3 antibodies were added to cells at a 1:500 dilution and incubated for 1 hour. Cells were washed with PBS supplemented with 5% BSA, and FITC-conjugated goat anti-rabbit antibodies were added and incubated for 30 minutes. Cells were washed with BSA-PBS and examined using fluorescence microscopy. For FGF1 analysis, cells were treated with 200 μM TM or 10 μl PBS for 24 hours, formalin fixed, treated with goat anti-human FGF1 antibodies at a 1:200 dilution and incubated for 4 hours. Cells were washed with PBS supplemented with 5% BSA, and Texas red-conjugated donkey anti-goat antibodies were added and incubated for 1 hour. Cells were washed with BSA-PBS and examined using fluorescence microscopy.

To determine occludin and ZO-1 expression patterns, cells were plated on transwells as described for TER assays. Media from the basolateral chamber was removed every other day and replaced with new media. Resistances were measured for 10 days, and either 100 ng/ml FGF2, 100 ng/ml FGF7 or 10 μl PBS were added on days 10 and 12. On day 15, media was removed from both the apical and basolateral chambers, and the apical chamber was washed twice with PBS. One ml of 1% (w/v) paraformaldehyde in cacodylate buffer was added to each well, the plates were incubated for 10 minutes at room temperature, and the wells were washed once in PBS. One ml permeabilization media, consisting of 0.2% Triton X-100 and 2% BSA in PBS, was added to each well. After a 10 minute incubation at room temperature, wells were washed three times. 20 μl primary antibodies (rabbit polyclonal anti-human or occludin rabbit polyclonal anti-human ZO-1) diluted 1:20 in 1% goat serum, 0.25% sodium azide in PBS, were added to the apical chamber of three wells per antibody per ligand, and plates were incubated for 1 hour in a humidified chamber. Wells were washed in PBS for 10 minutes at room temperature, and 20 μl goat anti-rabbit Texas red diluted 1:200 in 1% goat serum, 0.25% sodium azide in PBS was added to each well. After a 30 minute incubation in a dark humidified chamber, wells were washed three times in PBS. Membranes were carefully removed from transwells and placed on coverslips. 10 μl polyvinyl alcohol was added to each membrane, and the membranes were covered. Staining was then visualized by fluorescence microscopy. Controls of no antibody, primary antibody only, and secondary antibody only were performed. A Nikon HB10101AF fluorescence microscope was used to capture images at room temperature using a Hitachi HVC20 camera and Scion image acquisition software.

### Statistical Analysis

Results are expressed as mean±standard deviation. The Student's t test was used for statistical analysis. A P value of <0.05 was considered statistically significant.

### Results

### Caco-2 and T84 Cells Express FGFR3b

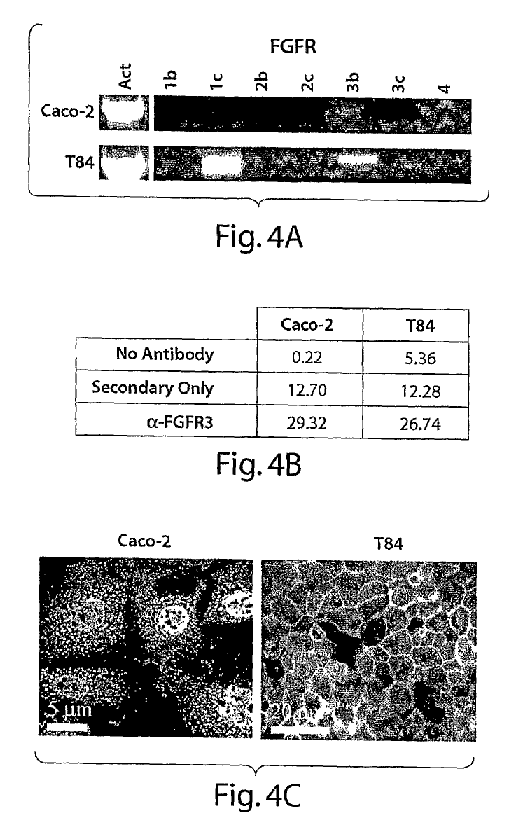

In order to explore FGF activity on colonic epithelial cells, the FGFR expression profile was first characterized. RT-PCR was performed for FGFR1b, 1c, 2b, 2c, 3b, 3c and 4, on Caco-2 cells. The primer pairs used to detect each isoform were demonstrated to specifically detect that FGFR isoform and not show cross-reactivity with other isoforms using BaF3 cells specifically transfected with each FGFR [36,37]. Both Caco-2 and T84 cells expressed FGFR3b (FIG. 4A). Caco-2 cells also potentially expressed FGFR4, and T84 cells expressed FGFR1c. FGFR4, however, was not expressed by T84 cells and FGFR1c was not expressed by Caco-2 cells. The expression of the FGFR3b protein in both Caco-2 and T84 cells was confirmed by FACS (FIG. 4B) as well as by immunohistochemistry (FIG. 4C). While the percentage of positive cells in FACS analysis did not correlate with the staining pattern observed with immunohistochemistry, this result is likely due to substantial digestion of cell surface proteins with the long incubation in trypsin required to detach the cells from culture flasks. FGFR3b expression was observed nonetheless using both methods. Additionally, Caco-2 cells have been previously demonstrated to express FGFR3b [27].

### FGF2 and FGF7 Induce Cellular Mediated Responses in Caco-2 and T84 Cells

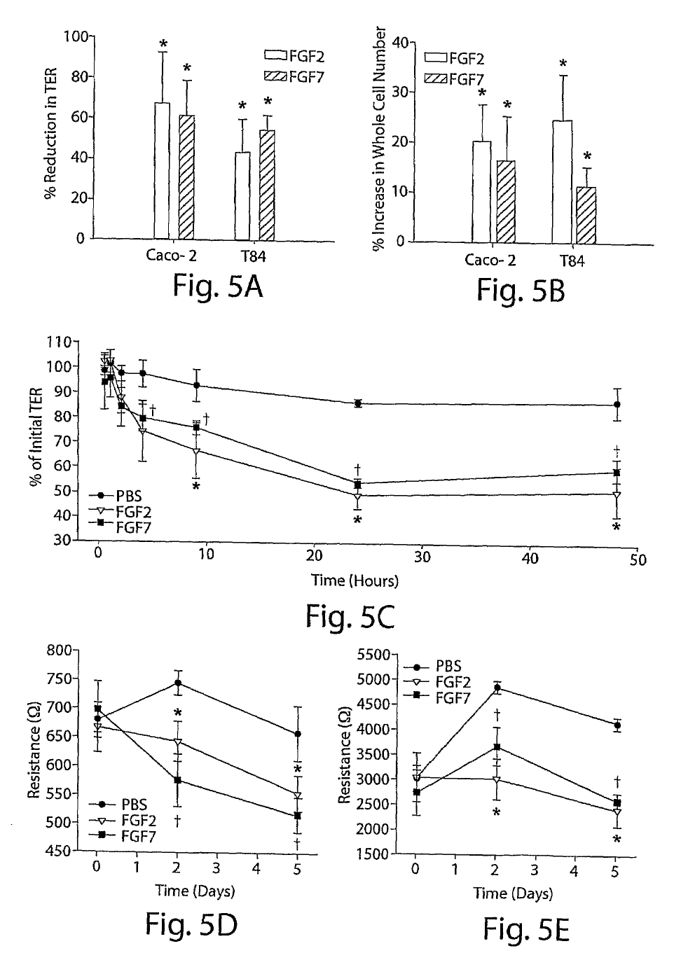

Both FGF2 and FGF7 promote cellular mediated activity in intestinal epithelial cells, which has been associated with FGFR3b [24,27]. The effects of FGF2 and FGF7 were investigated on intestinal epithelial cells using TER alterations as an output. TER is a measure associated in monolayer formation and integrity [34]. Caco-2 and T84 cells form monolayers with epithelial resistance mediated by tight junctions (TJs) [38,39]. Both FGF2 and FGF7 significantly reduced TER (FIG. 5A). FGF2 inhibited TER increases by 67.2±25.8% (p<0.05) in Caco-2 cells and by 43.4±16.6% (p<0.04) in T84 cells. FGF7 inhibited TER increases by 61.4±17.3% (p<0.02) in Caco-2 cells and by 54.6±7.6% (p<0.02) in T84 cells. The capacity of FGF2 and FGF7 to induce cellular mediated responses in Caco-2 and T84 cells was confirmed by measuring whole cell proliferation (FIG. 5B). FGF2 increased whole cell number by 20.2±7.4% (p<0.0003) in Caco-2 cells and by 24.8±8.8% (p<5×10⁻⁵) in T84 cells. FGF7 increased whole cell number by 16.4±8.9% (p<0.003) in Caco-2 cells and by 11.4±3.8% (p<0.006) in T84 cells.

The ability of FGF2 and FGF7 to reduce TER was further examined by observing its effects over different time scales. FGF2 and FGF7 reduced TER substantially after 24 hours and 48 hours in both Caco-2 cells (FIG. 5C) and T84 cells. With FGF2 and FGF7 were initially and again after 2 days, and with TER measured initially, at day 2, and at day 5, the ability of FGF2 and FGF7 to reduce TER was evident after both day 2 and day 5 in both Caco-2 cells (FIG. 5D) and T84 cells (FIG. 5E). On day 5, FGF2 and FGF7 reduced resistance by 43.6±11.1% (p<0.007) and 44.4±8.7% (p<0.003), respectively, compared to untreated Caco-2 cells and 41.7±8.1% (p<0.007) and 37.3±3.5% (p<0.0002), respectively, compared to untreated T84 cells. The effect of the two ligands was not significantly distinct in either Caco-2 cells (p>0.93) or T84 cells (p>0.45).

### FGF2 and FGF7 Promote Epithelial Monolayer Dysfunction

FGF2 and FGF7 promoted both TER inhibition and whole cell proliferation. The reduction in TER could, therefore, be attributed to increases in paracellular flux. To confirm the observed cellular mediated effects induced by FGF2 and FGF7, paracellular flux was measured using a 3 kDa dextran conjugated to FITC (Table 1). FGF2 and FGF7 both significantly increased flux in Caco-2 cells compared to PBS treated monolayers (p<0.003). FGF7 increased flux significantly more than FGF2 (p<0.04). Similarly, FGF2 and FGF7 both significantly increased flux in T84 cells (p<0.03).

TABLE 1
Flux across Caco-2 and T84 monolayers

| | PBS | FGF2 | FGF7 |
|---|---|---|---|
| Caco-2 | 20.28 ± 4.72 | 47.82 ± 4.64 | 117.14 ± 25.36 |
| T84 | 7.99 ± 0.22 | 37.21 ± 10.25 | 33.39 ± 9.24 |

Monolayers were grown for 15 days. 100 ng/ml ligand (FGF2 or FGF7) or 10 μl PBS was added on days 10 and 12. Flux was measured by the passage of 3 kDa-dextran conjugated to FITC on day 15. Data are reported as a function of emission at 530 nm over time, averaged over three experiments.

The increases in paracellular flux after ligand addition are consistent with TER decreases associated with cell separation. To confirm this, immunostaining for occludin (FIG. 3) and ZO-1, which are both components of TJs, was performed. Application of either FGF2 or FGF7 induced ruffling of cell membranes in both Caco-2 and T84 cells, consistent with alterations to TJs.

FGF2 and FGF7 reduced TER, increased paracellular flux and induced membrane ruffling. These findings are consistent with disruption of cell-cell contacts, reducing the total monolayer resistance and integrity [40]. Cell-cell contacts are mediated through apically located TJs and adjacent adherens junctions (AJs) [41]. Both types of junctions are important in regulating monolayer integrity, paracellular permeability, cell proliferation and cell differentiation [42-44]. TJs and AJs can be broken down, internalized and reorganized in response to extracellular cues including proliferative, inflammatory and pathological stimuli [42,45,46]. The reduction of cell-cell contacts observed in response to FGF2 and FGF7 suggests an alteration in the properties of TJs and AJs. Immunohistochemistry for occludin and ZO-1 confirmed that the cellular effects of FGF2 and FG7 involved TJs. TJs, however, can be altered without effects observed in AJs [47]. This study provides a demonstration that FGF family members can alter TJs. The ability of FGFs to affect TJs is consistent with their capacity to enable proliferation, migration and cell-cell interactions, all of which are important not only in maintaining and restoring the colonic epithelium, but also in angiogenesis.

### FGFR3b is Necessary for FGF2 and FGF7 to Reduce TER

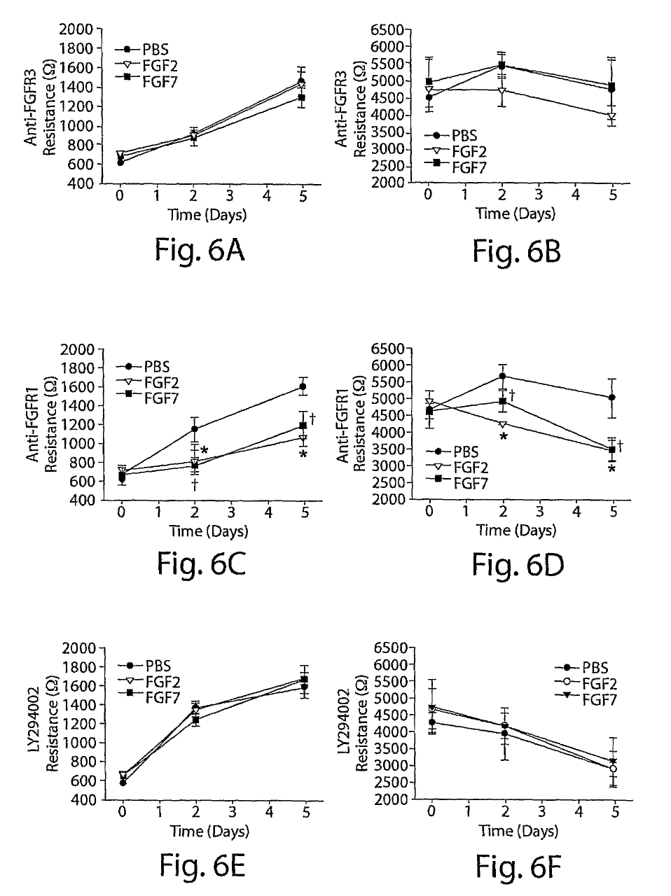

The cellular mediated effects of FGF2 and FGF7 have been associated with FGFR3b [27]. Therefore, whether FGFR3b was necessary for FGF2 and FGF7 to reduce TER was investigated. Antibodies to FGFR3 prevented both FGF2 and FGF7 from reducing TER in both Caco-2 cells (FIG. 6A) and T84 cells (FIG. 6B). The same antibody was previously confirmed to inhibit responses through FGFR3b in transfected BaF3 cells. Antibodies to FGFR1, however, did not prevent FGF2 or FGF7 from reducing TER in either Caco-2 cells (FIG. 6C) or T84 cells (FIG. 6D). The importance of the FGFR3b receptor was further examined using LY294002 to inhibit phosphoinositol 3-kinase (PI3K), which is downstream of FGFRs [48]. Treatment with LY294002 eliminated the ability of FGF2 and FGF7 to reduce TER in Caco-2 cells (FIG. 6E) and T84 cells (FIG. 6F). Inhibition of Mek/Erk with PD98059 and inhibition of p38 with SB203580, failed to eliminate the ligand mediated inhibition of TER. Mek/Erk activity is associated with the activities of FGF2 via FGFR1 [36, 49].

### FGF1 Reduces TER in Caco-2 and T84 Cells

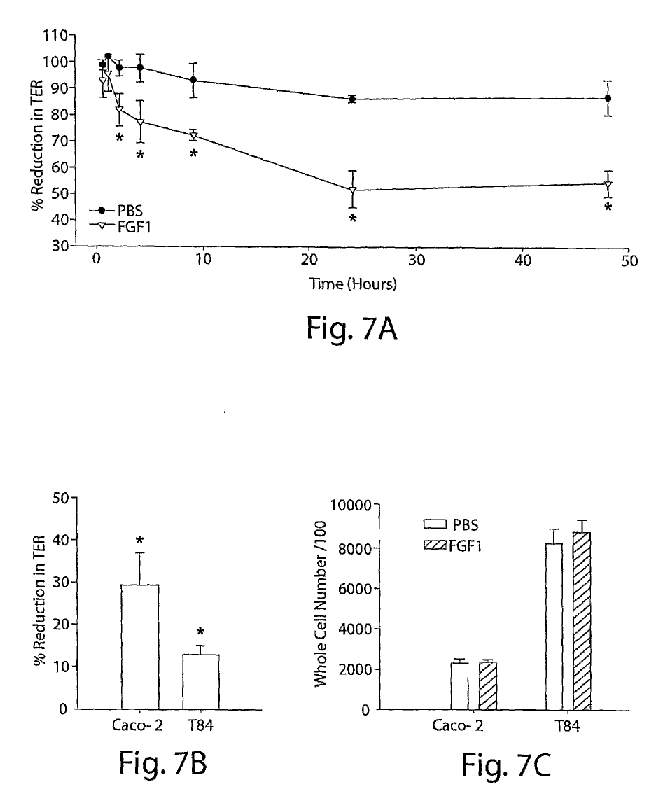

While FGFR3b is necessary for FGF2 and FGF7 to reduce TER, neither FGF2 nor FGF7 supports mitogenesis through this receptor [29]. FGF1, however, can support mitogenesis through FGFR3b [29]. Whether FGF1 could induce a biological response in Caco-2 cells and T84 cells was, therefore, investigated. FGF1 reduced TER in Caco-2 cells (FIG. 7A) and T84 cells over 48 hours similar to FGF2 and FGF7. At 48 hours, FGF1 reduced TER by 45.1±5.2% (p<0.004) in Caco-2 cells and by 22.7±3.2% (p<0.0008) in T84 cells. Over three days, FGF1 inhibited increases in TER by 35.1±12.3% (p<0.006) in Caco-2 cells and by 30.1±1.5% (p<0.0001) in T84 cells (FIG. 7B). FGF1 also significantly reduced TER after day 0 and day 2 doses and on day 5 in both Caco-2 (p<0.009) and T84 cells (p<0.006). Boiled FGF1 did not reduce TER. FGF1, however, did not induce the proliferation of either Caco-2 cells (p>0.68) or T84 cells (p>0.11) over three days (FIG. 7C).

FGF1, FGF2 and FGF7 have Identical Receptor Dependence in Caco-2 and T84 Cells

Whether FGF1 reduced TER through FGFR3b was examined. Antibodies to FGFR3, but not those to FGFR1, prevented reductions in TER after FGF1 administration. Furthermore, the use of the PI3K inhibitor LY294002 eliminated the ability of FGF1 to reduce TER. These results recapitulated those observed with FGF2 and FGF7, demonstrating that FGF1, FGF2 and FGF6 all required FGFR3b to induce a cellular mediated response.

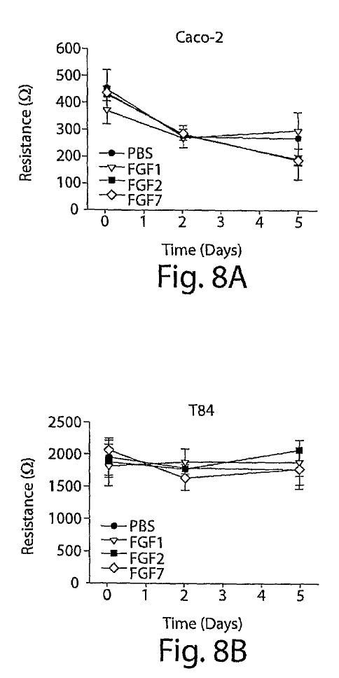

In addition to the high-affinity cell surface tyrosine kinase receptor, FGF activity is also influenced by low-affinity HSGAG receptors [8,11,29]. Therefore, whether FGF2 and FGF7 have the same low-affinity receptor requirements to induce a cellular mediated response as FGF1 was examined. Pretreatment of cells with sodium chlorate, which prevents heparan sulfate biosynthesis [50,51], was used to investigate the role of cell surface HSGAGs in conferring reductions in TER. Sodium chlorate treatment eliminated the ability for FGF1 (p>0.69 for Caco-2 cells; p>0.62 for T84 cells), FGF2 (p>0.34 for Caco-2 cells; p>0.22 for T84 cells) and FGF7 (p>0.28 for Caco-2 cells; p>0.97 for T84 cells) to reduce TER in Caco-2 cells (FIG. 8A) and T84 cells (FIG. 8B). FGF1, FGF2 and FGF7, therefore, have the same high-affinity and low-affinity receptor requirements to promote epithelial monolayer dysfunction.

The requirement of cell surface HSGAGs for FGF1, FGF2 and FGF7 activity is consistent with the possibility of syndecans serving an important role in FGF2 and FGF7 inducing a cellular response even in the absence of a cell surface receptor that could support their activity. Syndecans are a family of HSPGs that interact with ligands, including FGFs, through their core proteins and HSGAG side chains [52]. Syndecan clustering and associated actin stress fiber formation reduces monolayer integrity in endothelial cells [34]. The conserved cytoplasmic domains of syndecans interact with PDZ proteins including ZO-1 [52-55]. Correspondingly, both FGF2 and FGF7 do alter the distributions of the TJ proteins occludin and ZO-1 (FIG. 3).

### FGF1 is Necessary for FGF2 and FGF7 to Induce Cellular Mediated Responses

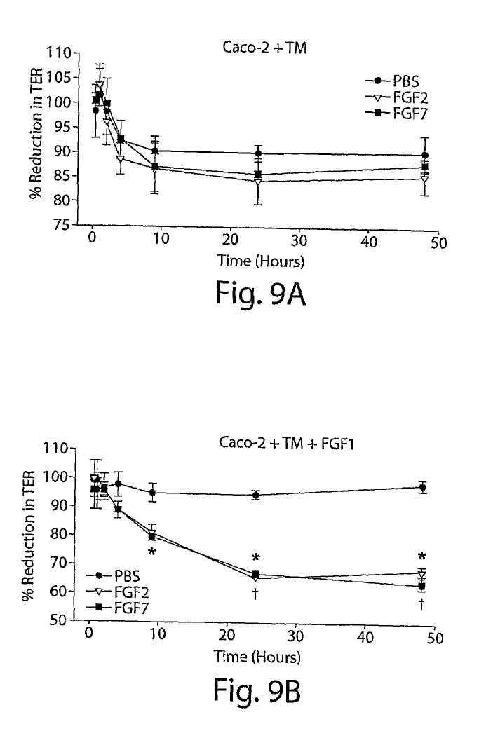

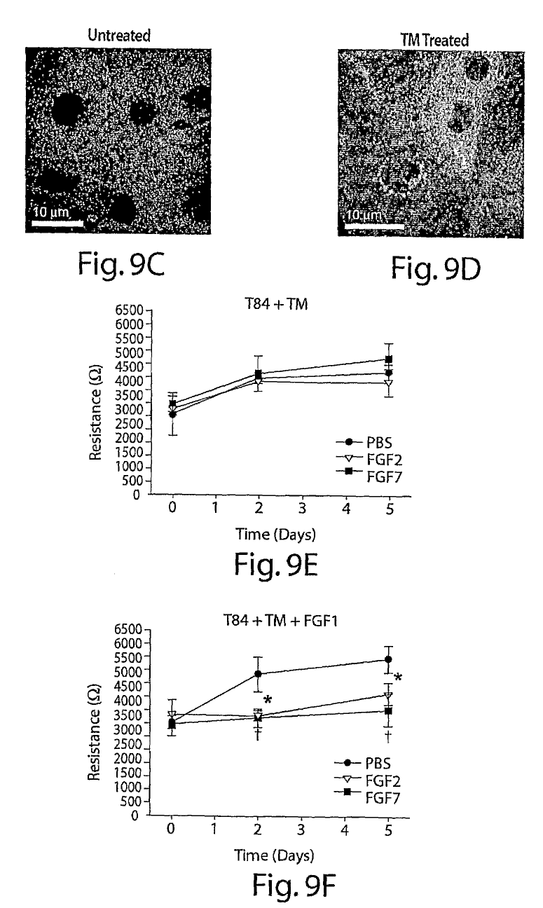

Whether FGF1 is essential for other FGFs to promote a cellular mediated response was investigated. Treating cells with TM, which inhibits the release of endogenous FGF1 [3], abrogated the reduction in TER induced by FGF2 (p>0.31) and by FGF7 (p>0.12) in Caco-2 cells (FIG. 9A). The addition of exogenous FGF1, however, restored the ability of FGF2 and FGF7 to reduce TER in TM treated Caco-2 cells (FIG. 9B). FGF2 elicited significant reduction in TER with FGF1 treatment after 9 hours (p<0.05), 24 hours (p<0.03) and 48 hours (p<0.005). FGF7 elicited significant reduction in TER after 24 hours (p<0.004) and 48 hours (p<0.004).

These results suggest that FGF1 is necessary for FGF2 and FGF7 to induce a cellular mediated response. In order for FGF1 to be present for FGF2 and FGF7 to act, it must be produced by the Caco-2 and T84 cells themselves. Therefore, whether Caco-2 and T84 cells expressed FGF1 was explored. Expression of FGF1 by Caco-2 cells (FIG. 9C) and T84 cells was confirmed by immunohistochemistry. Treating cells with TM alters the distribution of FGF1 with Caco-2 cells (FIG. 9D) and T84 cells, retaining FGF1 closer to the nucleus, consistent with TM preventing the release of FGF1 [3] in Caco-2 and T84 cells.

The ability of TM to eliminate reductions in TER induced by FGF2 and FGF7 also extended to T84 cells. TM prevented FGF2 (p>0.25) and FGF7 (p>0.23) from reducing TER in T84 cells (FIG. 9E). Additionally, FGF1 treatment restored TER reductions after 2 days (p<0.05 for FGF2; p<0.04 for FGF7) and 5 days (p<0.03 for FGF2; p<0.02 for FGF7) for both FGF2 and FGF7 (FIG. 9F).

To validate that extracellular FGF1 is essential for FGF2 and FGF7 to reduce TER, cells were treated with antibodies to FGF1. The treatment of Caco-2 cells and T84 cells with antibodies to FGF1 also prevented FGF2 (p>0.13 for Caco-2 cells; p>0.23 for T84 cells) and FGF7 (p>0.08 for Caco-2 cells; p>0.93 for T84 cells) from reducing TER. In order to further confirm the requirement of FGF1 for FGF2 and FGF7 cellular mediated responses, the effects of TM on whole cell proliferation was examined. TM eliminated the proliferative effect of FGF2 and FGF7 in both Caco-2 cells and T84 cells, while the addition of FGF1 again restored the proliferative capacity of these ligands in both cell types.

Eliminating FGF1 from the extracellular matrix with TM treatment [3], or neutralizing its activity, prevented FGF2 and FGF7 from eliciting a cellular mediated response. Inhibition of components of the FGF1 pathway—the ligand, the receptor or the downstream signal cascade—abrogated the ability of FGF2 and FGF7 to induce biological activity. Subsequently restoring FGF1 to the extracellular matrix enabled FGF2 and FGF7 to again impact cell function. FGF1 is therefore necessary and sufficient for FGF2- and FGF7-mediated activity in both Caco-2 and T84 cells.

Although FGF1-FGFR3b interactions are essential for FGF2 and FGF7 to induce cellular mediated responses, the mechanism for this activity is not clear. One potential mechanism is FGF1 activity through FGFR3b enabling syndecan clustering by other FGFs. FGF2 can induce cellular responses independent of FGFRs, through interactions with and internalization by HSPGs [56,57]. The dependence of FGF2 and FGF7 on FGFR3b would, therefore, be for the requisite FGF1 signaling. Syndecans serving an important role for FGF2 and FGF7 activity is consistent with the alterations in occludin and ZO-1 distributions [52-55]. Furthermore, this mechanism is also supported by the ability of FGF2 and FGF7 to induce proliferation, unlike FGF1.

### Therapeutic Implications

The therapeutic role of FGFs in IBD has been investigated. Each of FGF2, FGF7, FGF10 and FGF20 have shown some therapeutic benefit in attenuating the disease [5, 13-15]. The promising results observed in culture and in animal studies, however, have not been recapitulated in human studies [30-32]. This study sought to better understand the cellular mechanisms by which FGF family members induce cellular responses in the colon in order to shed insight into why FGFs have not had clinical success in treating IBD.

FGF1 is expressed by the normal intestinal epithelium and overexpressed in many colorectal adenomas and most colorectal cancers [58]. FGF1 autocrine signals promote epithelial cell survival [59]. The release of FGF2 and FGF7 enables protective and reparative functions by promoting angiogenesis, proliferation and migration [17,60]. In the unaffected colon, FGF2 and FGF7 may maintain and heal the epithelium in an FGF1-dependent manner and directly promote an appropriate level of angiogenesis. In IBD, FGF2 levels increase correlated to the level of disease [13] and FGF7 is significantly elevated [25]. If the levels of FGF1, which can inhibit inflammation, were to decrease, FGF2 and FGF7 would retain their angiogenic role but potentially lose their ability to reduce inflammation and maintain the epithelium. The activity of FGFs in IBD has correspondingly been associated with increasing colonic angiogenesis as well as transmural wall thickness [17,18]. FGF1 expression by colonic epithelial cells may serve as an important switch, defining whether colonic FGFs inhibit inflammation or promote angiogenesis.

The activity of FGFs is typically associated with the dimerization of cell surface tyrosine kinase receptors. The activity of FGF2 and FGF7 was found to be dependent on FGF1, FGFR3b and cell surface HSGAGs. FGF1 signaling through FGFR3b may enable the clustering of syndecans by FGF2 and FGF7. Consistent with this hypothesis, TJ protein distribution is altered by FGF2 and FGF7 signaling in this cell system, demonstrating for the first time that TJs can be altered by FGFs. Demonstrated herein is the importance of FGF1 for the activity of other FGFs with known therapeutic roles in IBD. Furthermore, this study highlights the role of syndecans in regulating inflammation in pathological settings such as IBD.

### Example 2

### Syndecan-1 is Protective in Inflammatory Bowel Disease

### Materials and Methods

### Cell Culture

Caco-2 cells (American Type Culture Collection, Manassas, Va.) were maintained in minimal essential medium (MEM; GibcoBRL, Gaithersburg, Md.) supplemented with 10% FBS (Hyclone, Logan, Utah), 15 mM HEPES, 100 mg/L penicillin and 100,000 U/L streptomycin (GibcoBRL). Cells were grown in 175 cm² flasks at 37° C. in a 5% CO₂ humidified incubator. Caco-2 cells were passaged twice a week at confluence. T84 cells were passaged once a week, at confluence.

### RT-PCR

Five μg of total RNA was isolated from Caco-2 cells using Trizol reagent (Life Tech, Rockville, Md.) followed by reverse transcription with random hexamers. Specific oligomers were designed based on published sequences in order to detect the expression of specific human Wnt, Fz and syndecan isoforms. Sequences of primer pairs were as follows: Wnt-1: 5′-CAC GAC CTC GTC TAC TTC GAC-3′ (SEQ ID NO: 17) and 5′-ACA GAC ACT CGT GCA GTA CGC-3′ (SEQ ID NO: 18) [88]; Wnt-2: 5′-CAT GGT GGT ACA TGA GAG CTA C-3′ (SEQ ID NO: 19) and 5′-GGC AAA TAC AAC TCC AGC TGA G-3′ (SEQ ID NO: 20) [89]; Wnt-3: 5′-GAG AGC CTC CCC GTC CAC AG-3′ (SEQ ID NO: 21) and 5′-CTG CCA GGA GTG TAT TCG CAT C-3′ (SEQ ID NO: 22) [90]; Wnt-3a: 5′-CAG GAA CTA CGT GGA GAT CAT G-3′ (SEQ ID NO: 23) and 5′-CCA TCC CAC CAA ACT CGA TGT C-3′ (SEQ ID NO: 24) [90]; Fz-1: 5′-GAA CTT TCC TCC AAC TTC ATG GC-3′ (SEQ ID NO: 25) and 5′-CAT TTC CAT TTT ACA GAC CGG-3′ (SEQ ID NO: 26) [91]; Fz-2: 5′-GGT GAG CCA GCA CTG CAA GAG-3′ (SEQ ID NO: 27) and 5′-CCT AAA AGT GAA ATG GTT TCG ATC G-3′ (SEQ ID NO: 28) [91]; Fz-10: 5′-ACA CGT CCA ACG CCA GCA TG-3′ (SEQ ID NO: 29) and 5′-ACG AGT CAT GTT GTA GCC GAT G-3′ (SEQ ID NO: 30) [91]; and Syndecan-1/Sdc-1/CD138: 5′-CTT CAC ACT CCC CAC ACA GA-3′ (SEQ ID NO: 31) and 5′-TCC TGT TTG GTG GGC TTC TG-3′ (SEQ ID NO: 32) [92]. To control for total cell protein, RT-PCR was also performed on β-actin using the primers 5′-GCC AGC TCA CCA TGG ATG ATG ATA T-3′ (SEQ ID NO: 33) and 5′-GCT TGC TGA TCC ACA TCT GCT GGA A-3′ (SEQ ID NO: 34) [36]. PCR was performed using the Advantage-GC cDNA kit from Clontech as per manufacturer's instructions (Palo Alto, Calif.).

### Transepithelial Resistance

Caco-2 cells were grown to just prior to confluency. The apical chambers of transwell plates with 6.5 mm diameter and 3 μm pore size filters were pretreated with 50 μl of 45 μg/ml mouse recombinant collagen IV (BD Biosciences, Bedford, Mass.) in 60% ethanol 24 hours prior to plating. Plates were incubated overnight at room temperature. Just prior to confluency, cells were washed with 20 ml phosphate buffered saline (PBS) and treated with 3.5 ml trypsin-EDTA per 175 cm² flask for 20-60 min at 37° C., until cells detached. Cells were resuspended in 16.5 ml media appropriate to the cell type. Each apical chamber was supplemented with 82 μl cell solution. The basolateral chamber was filled with 1 ml propagation media. Filters were fed with new media every two days over the course of experiments.

Caco-2 cells were allowed to grow on transwells to form monolayers. Monolayer formation was examined every other day by light microscopy as well as by TER measurements using an EVOM ohmmeter (World Precision Instruments, Sarasota, Fla.). After monolayer formation, cells were treated with 100 ng/ml recombinant human FGF1 (Amgen, Thousand Oaks, Calif.), 100 ng/ml recombinant human FGF2 [1], 100 ng/ml recombinant human FGF7 (Sigma, St. Louis, Mo.), or an equivalent volume (10 μl) of PBS. TER measurements were made after each of 30 minutes, 1 hour, 2 hours, 4 hours, 9 hours, 24 hours and 48 hours.

To probe the mechanism by which various FGFs induced cellular mediated effects, various media treatments were employed. TM was added 24 hours prior to the addition of ligands to yield a final concentration of 200 μM, sufficient to prevent the release of FGF1 [3]. Sodium chlorate, which reduces cell HSPGs and the sulfation of associated HSGAGs, was added 24 hours prior to the addition of ligands to yield a final concentration of 50 mM [34,93]. Heparin (Celsus, Cincinnati, Ohio) was added at a concentration of 500 ng/ml concurrently with ligands. PMA (100 ng/ml; Sigma, St. Louis, Mo.), which activates PKC, and promotes syndecan shedding [75], was added 2 hours prior to ligands and again, 24 hours after. Goat anti-human Fz-1 (Santa Cruz Biotechnology, Santa Cruz, Calif.) was added with ligands at a 1:100 dilution. All experiments were performed in triplicate.

### Immunohistochemistry

Caco-2 cells were grown to just prior to confluency on collagen IV coated glass slides. Cells were treated with PBS, FGF1, FGF2 and FGF7, and were incubated at 37° C. in a 5% CO₂ humidified incubator for 1 hour, 4 hours, 24 hours or 48 hours, as noted. Cells were washed with PBS, fixed with 3.7% formalin for 10 minutes and washed again with PBS. Cells were treated with 0.1% Triton x-100 for 5 minutes to extract lipids. Cells were washed with PBS and preincubated with PBS supplemented with 1% bovine serum albumin. Primary antibodies were added for 4 hours at room temperature, and cells were washed with PBS. Primary antibodies used were as follows: rabbit anti-human ZO-1 (1:160; Zymed Laboratories, San Francisco, Calif.); mouse anti-human syndecan-1 (1:300; Santa Cruz Biotechnology); goat anti-human Fz-1 (1:125; Santa Cruz Biotechnology); rabbit anti-human caveolin-1 (1:250; Santa Cruz Biotechnology); goat anti-human glypican-1 (1:100; Santa Cruz Biotechnology); mouse anti-human E-cadherin (1:250; Zymed Laboratories); rabbit anti-human β-catenin (1:125; Cell Signaling Technology, Beverly, Mass.); and rabbit anti-human phospho-β-catenin (Ser33/37/Thr41; 1:125; Cell Signaling Technology, Beverly, Mass.).

Cells were then treated with secondary antibodies, Texas Red-labeled phalloidin (Molecular Probes, Eugene, Oreg.) or FLAER-FL1 (1:400; Protox Biotech, Victoria, BC, Canada) as appropriate for 1 hour in the dark at room temperature. Secondary antibodies used were as follows: FITC-labeled chicken anti-rabbit (for ZO-1; 1:100; Molecular Probes, Eugene, Oreg.); Texas Red-labeled goat-anti mouse (for syndecan-1; 1:400; Molecular Probes); FITC-labeled donkey anti-mouse (for syndecan-1; 1:400; Molecular Probes, Eugene, Oreg.); FITC-labeled rabbit anti-mouse (for syndecan-1 and E-cadherin; 1:500; Molecular Probes, Eugene, Oreg.); Texas Red-labeled rabbit anti-goat (for Fz-1; 1:200; Jackson ImmunoResearch Laboratories, West Grove, Pa.); Texas Red-labeled goat anti-rabbit (for caveolin-1, β-catenin and phospho-β-catenin; 1:500; Molecular Probes); and Texas Red-labeled donkey anti-goat (for glypican-1; 1:80; Research Diagnostics, Flanders, N.J.).

Cells, except those stained with phalloidin, were washed with PBS and stained with diamidino-2-phenylindole (DAPI) for 5 minutes at room temperature (Molecular Probes). Controls of no antibody, primary antibody only, and secondary antibody only were performed. Cells were examined by confocal microscopy (ZO-1 and phalloidin only), or by fluorescence microscopy. A Nikon HB10101AF fluorescence microscope was used and images were captured at room temperature using a Hitachi HVC20 camera and Scion image acquisition software. Images were processed using Adobe Photoshop 7.0 and Adobe Illustrator 10.0.

### TCF/LEF Assay

Caco-2 cells were seeded in 12 well plates such that they would reach 90% confluency in 4 days. Two days after plating, Caco-2 cells were transiently transfected with the TOPFLASH (contains 4 TCF/LEF binding elements) luciferase promoter construct (Promega, Madison, Wis.) as well as a thymidine kinase driven renilla luciferase construct (Promega, Madison, Wis.) as a transfection control vector. Each well was transfected with 1.5 μg DNA using Superfect transfection reagent (Promega, Madison, Wis.). Twenty-four hours after transfection, Caco-2 cells were treated with PBS, FGF1, FGF2 or FGF7, for 1 hour, 4 hours or 24 hours. The Caco-2 cells were processed using a dual-luciferase assay system to measure luciferase activity from both the TOPFLASH constructs and the renilla constructs. All results were standardized to the relative transfection efficiencies as observed from the renilla values.

### DSS Acute Colitis Model

Mice with knock-outs of Sdc-1, the gene for syndecan-1, were produced as previously described [66-68]. Disease was induced by ad libitum administration of 10% DSS in the drinking water [26]. Survival and the weight of surviving mice were measured over time. Ten wild-type and ten Sdc-1 knock-out mice were used for experiments.

### Statistical Analysis

Results are expressed as mean±standard deviation. The Student's t test was used for statistical analysis. A P value of <0.05 was considered statistically significant.

### Results

### FGF1 is Necessary for FGF2 and FGF7 to Reduce TER in Caco-2 Cells

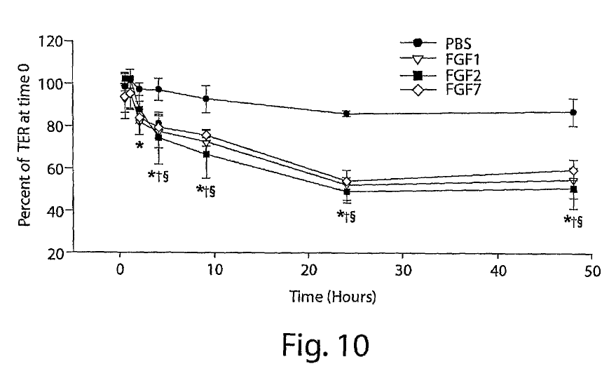

In order to explore how FGF1 enabled FGF2 and FGF7 activity in Caco-2 cells, the ability of each of the ligands to affect TER was first reproduced. Each of FGF1, FGF2 and FGF7 significantly reduced TER (FIG. 10). Reductions in TER were evident 2 hours after FGF1 treatment (p<0.03) and 4 hours after FGF2 or FGF7 treatments (p<0.05). Significant reductions were evident at all subsequent time points examined (p<0.04). The magnitude of TER reduction elicited by FGF1, FGF2 and FGF7, however, was not significantly different (p>0.20). Each of FGF1, FGF2 and FGF7 could, therefore, reduce TER.

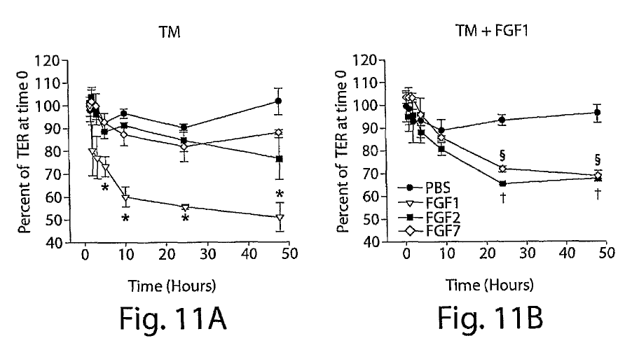

Ammonium tetrathiomolybdate (TM) was subsequently used to confirm the requirement of FGF1 for FGF2 and FGF7 to reduce TER. Treating cells with TM, which inhibits the release of endogenous FGF1 [3], abrogated the reduction in TER induced by FGF2 (p>0.13) and by FGF7 (p>0.17) at all time points (FIG. 11A). FGF1, however, still elicited significant reductions in TER with TM treatment at all time points beginning at 4 hours (p<0.05). The addition of exogenous FGF1, however, restored the ability of FGF2 and FGF7 to reduce TER in TM treated Caco-2 cells (FIG. 11B). FGF2 and FGF7 both significantly reduced TER after 24 hours (p<0.04) and 48 hours (p<0.04). Since supplementing TM-treated cells with FGF1 cells reduces TER, the effects of FGF2 and FGF7 in the presence of TM and FGF1 were compared to TM-treated cells. These results showed that FGF1 is necessary for FGF2 and FGF7 to reduce TER in Caco-2 cells.

### Cell Surface HSPGs are Necessary for FGF2 and FGF7 to Reduce TER

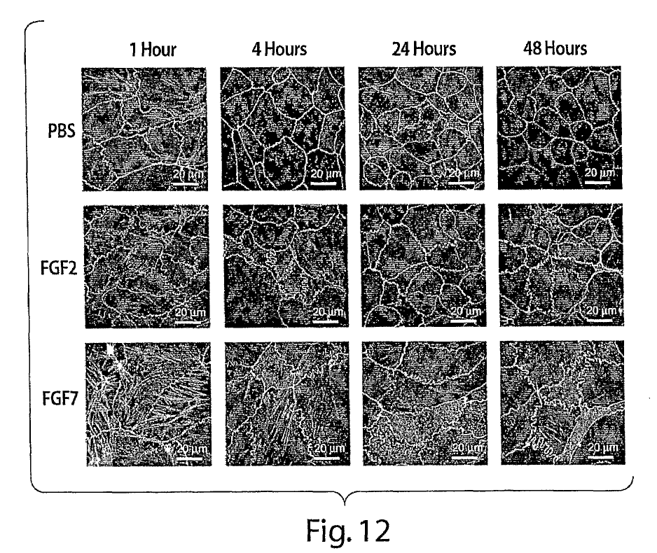

FGF-induced TER reductions were demonstrated to result from increased paracellular flux and reduced cell-cell contacts. To examine the effects of FGFs on cell-cell interactions over time, immunostaining for the tight junction (TJ) protein ZO-1 was performed (FIG. 12). FGF2 and FGF7 induced notable membrane ruffling at 1 hour, 4 hours, 24 hours and 48 hours, confirming that the reductions in TER over time are associated with disruption of cell-cell contacts.

Cell-cell contacts are formed by both apical TJs and adjacent adherens junctions (AJs) [73]. While both TJs and AJs are important in regulating monolayer integrity, TJs interact with cell surface HSPGs. Specifically, ZO-1 and other PDZ proteins interact with the conserved cytoplasmic domains of syndecans, a family of HSPGs [54,55,69,70]. As the HSGAG component of HSPGs interacts with FGFs, cell surface syndecans offer a potential mechanism by which FGFs affect TJs and, therefore, TER.

The ability of FGF2 and FGF7 to promote actin stress fiber formation was additionally investigated. Both FGF2 and FGF7 induced actin stress fiber formation (FIG. 12). Monolayer integrity can be reduced by syndecan clustering and associated actin stress fiber formation [34]. The ability of FGF2 and FGF7 to reduce TER, alter TJ proteins, such as ZO-1, and induce stress fiber formation is consistent with a syndecan-mediated mechanism.

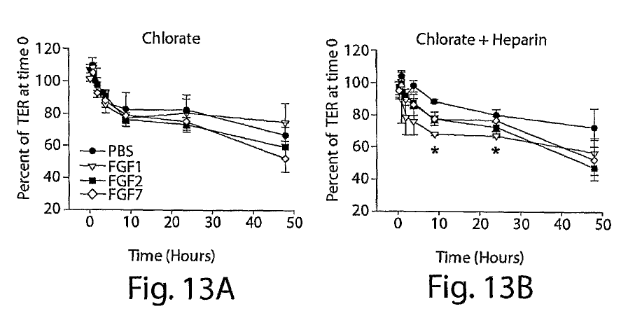

Cell surface HSPGs, including syndecans, are protein-HSGAG conjugates [74]. To investigate the importance of the role of the HSGAG component of cell surface HSPGs in mediating TER reductions, cells were pretreated with sodium chlorate, which prevents heparan sulfate biosynthesis [50,51]. Sodium chlorate treatment abrogated the ability of FGF1 (p>0.49), FGF2 (p>0.38) and FGF7 (p>0.16) to reduce TER compared to PBS treatment (FIG. 13A). With the addition of heparin (FIG. 13B), FGF1 reduced TER compared to PBS at 9 hours (p<0.003) and at 24 hours (p<0.004). The ability of FGF2 (p>0.08) and FGF7 (p>0.16) to reduce TER, however, was not restored with heparin. While HSGAGs were necessary for each of FGF1, FGF2 and FGF7 to reduce TER, the requirements for FGF1 differed from those of FGF2 and FGF7. HSGAGs free in the extracellular matrix were not sufficient for FGF2 and FGF7 to reduce TER, but were for FGF1. These findings suggest that the HSGAG component of cell surface HSPGs is necessary for FGF2 and FGF7 to reduce TER.

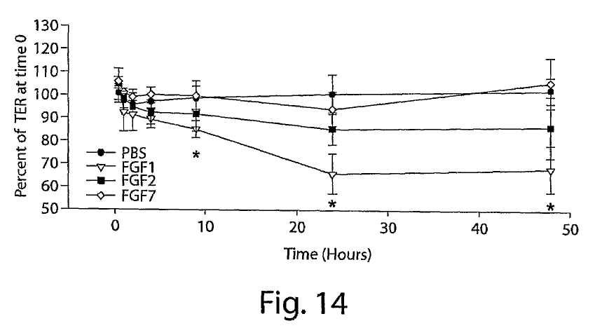

To verify the importance of cell surface HSPGs, Caco-2 cells were treated with phorbol 12-myristate 13-acetate (PMA), which activates protein kinase C (PKC), and as a result, accelerates the shedding of syndecan-1 and syndecan-4 [75]. After PMA treatment (FIG. 14), FGF1 reduced TER after 9 hours (p<0.03), 24 hours (p<0.008) and 48 hours (p<0.02). Neither FGF2 (p>0.07) nor FGF7 (p>0.35) reduced TER in PMA treated Caco-2 cells at any time point. Loss of cell surface syndecan-1 and syndecan-4 by PMA-induced shedding therefore prevented FGF2 and FGF7 activity, but not that of FGF1.

These results demonstrate that cell surface HSPGs are necessary for FGF2 and FGF7 to reduce TER. The HSGAG component of the HSPG is necessary likely to mediate ligand binding [8]. The ability of FGF2 and FGF7 to induce membrane ruffling and promote actin stress fibers formation confirms the importance of the protein component of the HSPG as the cytoplasmic domains of syndecans interact with ZO-1 and other PDZ proteins [54,55,69,70]. The effects on ZO-1 and TJs additionally suggest that the involved HSPGs are most likely syndecans. Syndecan-1 and syndecan-4 clustering promotes actin stress fiber formation [34]. Furthermore, PMA promotes syndecan-1 and syndecan-4 shedding [75], preventing FGF2- and FGF7-mediated reductions in TER. Taken together, the necessary HSPG for FGF2 and FGF7 to reduce TER is syndecan-1 and/or syndecan-4.

### FGF2 and FGF7 Alter Syndecan-1 Localization

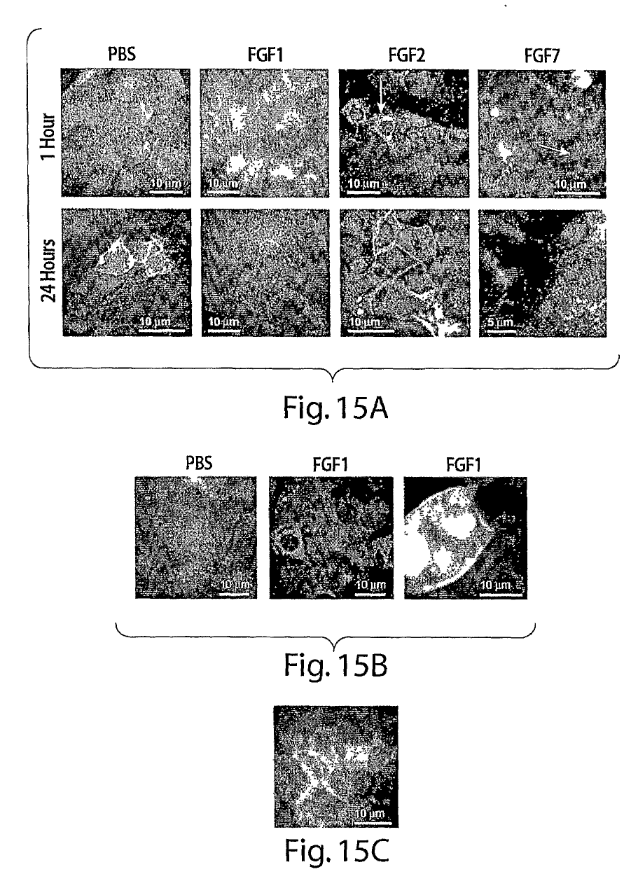

The effects of FGF1, FGF2 and FGF7 on syndecan-1 distribution were examined. One hour after FGF2 and FGF7 treatment, syndecan-1 was primarily localized around the nucleus (FIG. 15A). This finding was seen to a lesser amount with FGF1 treatment and was absent after PBS treatment. After 24 hours, FGF2 and FGF7 treated cells revealed a moderately enhanced cytoplasmic and membrane-associated distribution of syndecan-1 compared to FGF1 treated cells. Glypican-1, another HSPG, was not affected by FGF1, FGF2 or FGF7. The nuclear localization of syndecan-1 is of particular note as HSPGs have been hypothesized to serve as a shuttle to transport heparin-binding growth factors, specifically including FGF2, to the nucleus [72]. As a result, transport of syndecan-1 with bound growth factors could serve as a mechanism by which Caco-2 cells could respond to FGF2 and FGF7 even though the cell surface tyrosine kinase receptors cannot support a response from these ligands [29].

While syndecan-1 may enable FGF2 and FGF7 to induce a cellular response, FGF1 is necessary for FGF2 and FGF7 to reduce TER. One potential explanation is that FGF1 may activate processes that allow syndecan-1 to serve its role facilitating FGF2 and FGF7 activity. HSPGs can be localized within lipid rafts, creating high local concentrations of binding sites for heparin-binding growth factors such as FGF2, promoting ligand association and inhibiting dissociation [57]. The localization of caveolin-1, a marker for lipid rafts [76], after FGF1 treatment was investigated. PBS did not induce the nuclear localization of syndecan-1 or caveolin-1 (FIG. 15B). Four hours after administration of FGF1, caveolin-1 was localized surrounding the nucleus. Furthermore, caveolin-1 and syndecan-1 did co-localize near the nucleus.

To confirm the ability of FGF1 to promote syndecan-1 association with lipid rafts, immunohistochemistry was performed for syndecan-1 and glycosylphosphatidylinositol (GPI)-anchored proteins. FLAER, an Alexa-488 conjugated inactive variant of the protein proaerolysin, detects GPI-anchored proteins [77,78]. Syndecan-1 and GPI-anchored proteins were found to exhibit co-localization at the membrane and within the cytoplasm (FIG. 15C). The co-localization of syndecan-1 and GPI-anchored proteins is consistent with syndecan-1 in the cytoplasm originating from regions rich in lipid rafts [79].

The data presented reveal that FGF2 and FGF7 activity is associated with syndecan-1 translocation to the nucleus. Syndecan-1 may enable FGF2 and FGF7 reductions in TER by binding the ligands and shuttling them to the nucleus [72], serving to bypass the cell surface FGFRs that do not support their activity [29]. The ability of syndecan-1 to translocate FGF2 and FGF7 may be promoted by FGF1. FGF1 promotes the localization of lipid rafts to the nucleus, and syndecan-1 co-localizes with these lipid rafts. As such, FGF1 activity may cause syndecan-1 to move to the nucleus by causing lipid rafts to translocate them. FGF2 and FGF7, prior to this activity, could be bound by the HSGAG component of syndecan-1, and with FGF1 activity, they would be specifically induced to be brought to the nucleus, where they could initiate signaling.

### FGF2 and FGF7 Alter Wnt Signaling

Syndecan-1 is known to modulate Wnt signaling [68]. Wnt signaling is initiated by the Wnt ligand binding to Frizzled (Fz) protein receptors as well as lipoprotein receptor-related proteins [80]. Initiation of the Wnt cascade results in the cytoplasmic accumulation of β-catenin, which binds to a protein complex including T cell factor/lymphoid enhancer factor (TCF/LEF) transcription factors and induces gene expression [80,81]. To promote cellular responses, Wnt inhibits glycogen synthase-3β, thereby stabilizing β-catenin and causing its cytoplasmic localization from being sequestered by proteins including E-cadherin [80]. Slug/Snail family proteins, induced by signaling by a variety of growth factors including FGFs, inhibit E-cadherin transcription [82]. Their activity can reduce cell-cell adhesion concomitantly with the accumulation of cytoplasmic β-catenin [80,82].

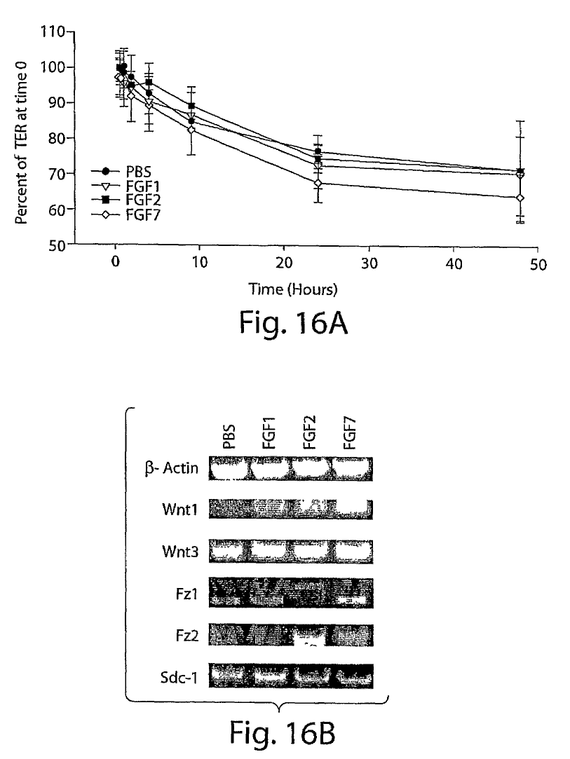

To investigate the potential involvement of the Wnt pathway, Caco-2 cells were supplemented with anti-Fz antibodies. Treating Caco-2 cells with anti-Fz antibodies prevented FGF1 (p>0.26), FGF2 (p>0.51) and FGF7 (p>0.16) from reducing TER (FIG. 16A). RT-PCR was performed to confirm the expression of members of the Wnt-signaling cascade. Specifically, RT-PCR demonstrated the expression of Wnt1, Wnt3, Fz1, Fz2 and syndecan-1 (the protein produce by the Sdc-1 gene; FIG. 16B). No expression of Wnt2, Wnt3A or Fz10 was detected by RT-PCR. The expression of Fz1 was further confirmed by immunohistochemistry. Of note, the RT-PCR data suggest that Wnt1 expression was increased with FGFs. FGF1, FGF2 and FGF7 all increased Wnt1 expression (Table 2). FGF2 and FGF7 also both increased Fz2 expression, while only FGF7 increased Fz1 expression.

TABLE 2
FGFs increase Wnt1 expression

| | PBS | FGF1 | FGF2 | FGF7 |
|---|---|---|---|---|
| Wnt1 | 1.0 | 2.2 | 2.7 | 5.1 |
| Wnt3 | 1.0 | 1.3 | 1.3 | 1.2 |
| Fz1 | 1.0 | 1.5 | 1.1 | 2.4 |
| Fz2 | 1.0 | 0.7 | 1.7 | 1.6 |
| Sdc-1 | 1.0 | 1.3 | 0.9 | 0.6 |

Intensity of RT-PCR images was quantified using NIH ImageJ. Data were normalized to the intensity of expression after PBS treatment, which was defined as 1.0.

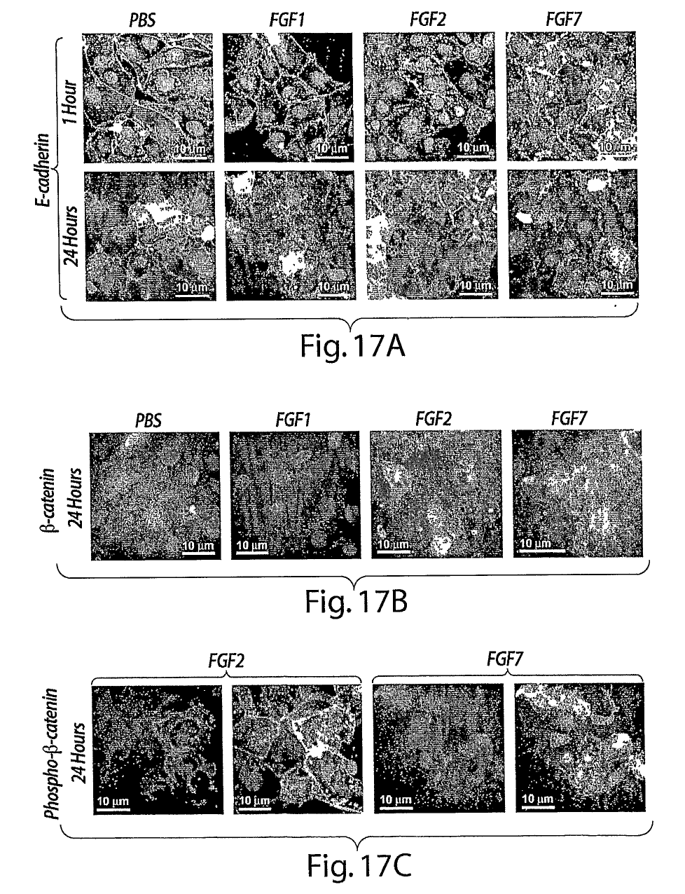

E-cadherin, associated with Wnt activities, is the prototypical and best characterized AJ protein [83]. Whether E-cadherin distribution was affected by FGFs was investigated. FGF2 and FGF7 altered E-cadherin localization after 1 hour (FIG. 17A), while PBS and FGF1 did not. After 24 hours, E-cadherin exhibited more cytoplasmic localization with FGF2 and FGF7 treatments than at 1 hour, and more than PBS and FGF1 treatment at 24 hours. FGF2 and FGF7 therefore affect AJs, suggesting that when these ligands are brought to the nucleus, they may activate distinct processes from FGF1. Furthermore, the Wnt pathway may be affected by FGF2 and FGF7.

To further probe the involvement of Wnt signaling, the effects of PBS, FGF1, FGF2 and FGF7 on β-catenin localization were examined. After 24 hours, FGF2 and FGF7 induced markedly increased cytoplasmic β-catenin compared to PBS treatment, which clustered around the nucleus (FIG. 17B). FGF1 treatment yielded potentially reduced levels of cytoplasmic β-catenin compared to PBS treatment. Phosphorylated β-catenin also clustered around the nucleus after Caco-2 cells were treated with FGF2 and FGF7 (FIG. 17C), which was not observed with PBS or FGF. Of note, much of the detected phosphorylated β-catenin appeared in distinct locations from E-cadherin, consistent with β-catenin being sequestered to the cytoplasm from E-cadherins. Furthermore, FGF2 and FGF7 increased TCF/LEF activity compared to PBS and FGF1. Taken together, these results suggest that FGF2 and FGF7 activity is associated with Wnt signaling, which supports the involvement of syndecan-1 in enabling FGF2 and FGF7 to promote cellular responses. Nonetheless, syndecan-4 may also facilitate FGF2 and FGF7 activity.

### Syndecan-1 is Protective in IBD

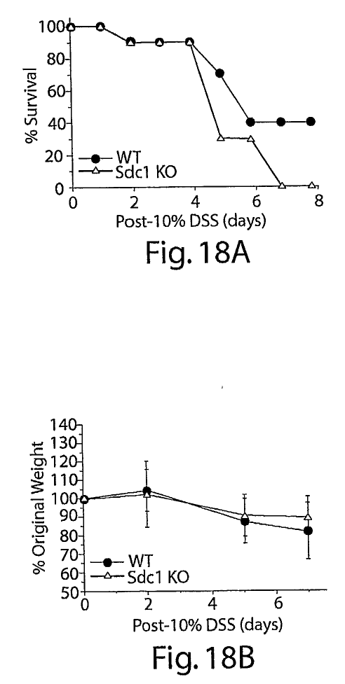

The data presented suggest that syndecan-1 enables FGF2 and FGF7 to reduce TER in Caco-2 cells even though they do not express the corresponding cell surface FGFRs. FGF1 uses a distinct pathway, most likely via FGFR3b, which is expressed by Caco2 cells [27], and which supports FGF1 activity [29]. Given that FGF2 and FGF7 promote intestinal epithelial cell growth, wound healing and reduce the inflammation associated with IBD [23,26,71], the protective effect of syndecan-1 in IBD was examined. Wild-type and Sdc-1 (the gene for syndecan-1) knock-out mice were administered drinking water with 10% dextran sodium sulfate (DSS) to induce an acute colitis. The DSS model promotes an acute disease that substantially affects survival. Sdc-1 knock-out mice, whose survival was not different from wild-type mice in an unmodified environment, were notably more susceptible to DSS than wild-type mice (FIG. 18A). In Sdc-1 knock-out mice, all mice had died by 7 days of ingesting DSS-water, while 40% of the wild-type mice survived for 8 days. The surviving mice exhibited no significant weight change over the course of the experiment or significant weight difference between the groups at any time point (FIG. 18B). Syndecan-1, therefore, appears to have a protective effect in acute IBD.

The data presented demonstrate that syndecan-1 is a specific cell surface HSPG that has a pivotal role in IBD. Syndecan-1 can enable the activity of FGFs in the absence of cognate cell surface FGFRs, likely by promoting ligand translocation to the nucleus [72]. Furthermore, the action of syndecan-1 is associated with alterations in cell-cell contacts through both TJs and AJs, which are important in processes essential for maintaining and healing the epithelia [84-86]. These activities, as well as effects on Wnt signaling, associated with syndecan-1 [68] present potential mechanisms by which this HSPG may induce protective effects in IBD. Methods and compositions are provided to enhance syndecan-1 expression for its potential in ameliorating aberrant inflammatory response, such as in IBD.

FGF2 and FGF7 induce cellular mediated responses in Caco-2 cells, which express a cell surface FGFR that does not support the activity of these FGFs. FGF1, however, was found to be necessary for FGF2 and FGF7 to induce a cellular mediated response. Provided herein is, therefore, the mechanism by which FGF1 enables FGF2 and FGF7 activity. The data presented demonstrate that FGF2 and FGF7 are dependent on cell surface HSPGs, while FGF1 is not. Syndecan-1 was found to translocate to the nucleus, a mechanism which has been previously validated by which FGFs can induce a cellular response. Of note, FGF1 promoted the clustering of syndecan-1 with lipid rafts, which together co-localize around the nucleus. This may, therefore, serve as the mechanism by which FGF1 enables FGF2 and FGF7 to induce cellular responses. Unlike FGF1, both FGF2 and FGF7 also affect the Wnt pathway, which has important implications in the colon, especially regarding cell transformation and tumor growth. Furthermore, these studies identified syndecan-1 as a potentially important modulator in IBD. Correspondingly, the presence of syndecan-1 was found to have a substantial protective effect in acute colitis. This study, therefore, demonstrates that syndecan-1 is a factor in IBD.

### REFERENCES

1. Kwan, C.-P., Venkataraman, G., Shriver, Z., Raman, R., Liu, D., Qi, Y., Varticovski, L., and Sasisekharan, R. (2001) Probing Fibroblast growth factor dimerization and role of heparin-like glycosaminoglycans in modulating dimerization and signaling. J Biol Chem 276, 23421-23429.
2. U.S. Patent Application 2003/0008820. Methods and products related to FGF dimerization. Kwan, C. P., Venkataraman, G., Shriver, Z., Raman, R., Berry, D., and Sasisekharan R.
3. Landriscina M, Bagala C, Mandinova A, Soldi R, Micucci I, Bellum S, Prudovsky I, Maciag T. (2001) Copper induces the assembly of a multiprotein aggregate implicated in the release of fibroblast growth factor 1 in response to stress. J Biol Chem 276, 25549-25557
4. U.S. Patent Application 2004/0071787. Pharmacologic, therapeutic and diagnostic regulation of FGF-1 export. Maciag, T., Hampton, B., Burgess, W. H., Gamble, S. M., Tarantini, F., and Jackson, A. Q.
5. Han, D. S., Li, F., Holt, L., Connolly, K., Hubert, M., Miceli, R., Okoye, Z., Santiago, G., Windle, K., Wong, E., and Sartor, R. B. (2000) Keratinocyte growth factor-2 (FGF-10) promotes healing of experimental small intestinal ulceration in rats. Am J Physiol Gastrointest Liver Physiol 279, G1011-1022.
6. Day, R., Ilyas, M., Daszak, P., Talbot, I., and Forbes, A. (1999) Expression of syndecan-1 in inflammatory bowel disease and a possible mechanism of heparin therapy. Dig Dis Sci 44, 2508-2515.
7. Uthoff, S. M., Eichenberger, M. R., Lewis, R. K., Fox, M. P., Hamilton, C. J., McAuliffe, T. L., Grimes, H. L., Galandiuk, S. (2001) Identification of candidate genes in ulcerative colitis and Crohn's disease using cDNA array technology. Int J Oncol 19, 803-810.
8. Rapraeger, A. C. (1993). The coordinated regulation of heparan sulfate, syndecans and cell behavior. Curr Opin Cell Biol 5, 844-853.
9. Sleeman, M., Fraser, J., McDonald, M., Yuan, S., White, D., Grandison, P., Kumble, K., Watson, J. D., and Murison, J. G. (2001). Identification of a new fibroblast growth factor receptor, FGFR5. Gene 271, 171-182.
10. Herr, A. B., Ornitz, D. M., Sasiseldlaran, R., Venkataraman, G., and Waksman, G. (1997). Heparin-induced self-association of fibroblast growth factor-2. Evidence for two oligomerization processes. J Biol Chem 272, 16382-16389.
11. Givol, D., and Yayon, A. (1992). Complexity of FGF receptors: genetic basis for structural diversity and functional specificity. Faseb J 6, 3362-3369.
12. Montesano, R., Vassalli, J. D., Baird, A., Guillemin, R., and Orci, L. (1986). Basic fibroblast growth factor induces angiogenesis in vitro. Proc Natl Acad Sci USA 83, 7297-7301.
13. Bousvaros, A., Zurkowski, D., Fishman, S. J., Keough, K., Law, T., Sun, C., and Leichtner, A. M. (1997). Serum basic fibroblast growth factor in pediatric Crohn's disease. Implications for wound healing. Dig Dis Sci 42, 378-386.
14. Jeffers, M., McDonald, W. F., Chillalcuru, R. A., Yang, M., Nakase, H., Deegler, L. L., Sylander, E. D., Rittman, R., Bendele, A., Sartor, R. B., and Lichenstein, H. S. (2002). A novel human fibroblast growth factor treats experimental intestinal inflammation. Gastroenterology 123, 1151-1162.
15. Chen, Y., Chou, K., Fuchs, E., Havran, W. L., and Boismenu, R. (2002). Protection of the intestinal mucosa by intraepithelial gamma delta T cells. Proc Natl Acad Sci USA 99, 14338-14343.
16. Beck, P. L., and Podolslcy, D. K. (1999). Growth factors in inflammatory bowel disease. Inflamm Bowel Dis 5, 44-60.
17. Kanazawa, S., Tsunoda, T., Onuma, E., Majima, T., Kagiyama, M., and Kikuchi, K. (2001). VEGF, basic-FGF, and TGF-beta in Crohn's disease and ulcerative colitis: a novel mechanism of chronic intestinal inflammation. Am J Gastroenterol 96, 822-828.
18. Di Sabatino, A., Ciccocioppo, R., Armellini, E., Morera, R., Ricevuti, L., Cazzola, P., Fulle, I., and Corazza, G. R. (2004). Serum bFGF and VEGF correlate respectively with bowel wall thickness and intramural blood flow in Crohn's disease. Inflamm Bowel Dis 10, 573-577.
19. Omata, F., Birkenbach, M., Matsuzaki, S., Christ, A. D., and Blumberg, R. S. (2001). The expression of IL-12 p40 and its homologue, Epstein-Barr virus-induced gene 3, in inflammatory bowel disease. Inflamm Bowel Dis 7, 215-220.
20. Finch, P. W., and Cheng, A. L. (1999). Analysis of the cellular basis of keratinocyte growth factor overexpression in inflammatory bowel disease. Gut 45, 848-855.
21. Folkman, J., Szabo, S., Stovroff, M., McNeil, P., Li, W., and Shing, Y. (1991). Duodenal ulcer. Discovery of a new mechanism and development of angiogenic therapy that accelerates healing. Ann Surg 214, 414-425; discussion 426-417.
22. Hull, M. A., Cullen, D. J., Hudson, N., and Hawkey, C. J. (1995). Basic fibroblast growth factor treatment for non-steroidal anti-inflammatory drug associated gastric ulceration. Gut 37, 610-612.
23. Szabo, S., Folkman, J., Vattay, P., Morales, R. E., Pinkus, G. S., and Kato, K. (1994). Accelerated healing of duodenal ulcers by oral administration of a mutein of basic fibroblast growth factor in rats. Gastroenterology 106, 1106-1111.
24. Dignass, A. U., Tsunekawa, S., and Podolsky, D. K. (1994). Fibroblast growth factors modulate intestinal epithelial cell growth and migration. Gastroenterology 106, 1254-1262.
25. Finch, P. W., Pricolo, V., Wu, A., and Finkelstein, S. D. (1996). Increased expression of keratinocyte growth factor messenger RNA associated with inflammatory bowel disease. Gastroenterology 110, 441-451.
26. Byrne, F. R., Farrell, C. L., Aranda, R., Rex, K. L., Scully, S., Brown, H. L., Flores, S. A., Gu, L. H., Danilenko, D. M., Lacey, D. L., Ziegler, T. R., and Senaldi, G. (2002). rHuKGF ameliorates symptoms in DSS and CD4(+)CD45RB (Hi) T cell transfer mouse models of inflammatory bowel disease. Am J Physiol Gastrointest Liver Physiol 282, G690-701.
27. Kanai, M., Rosenberg, I., and Podolsky, D. K. (1997). Cytokine regulation of fibroblast growth factor receptor 3 IIIb in intestinal epithelial cells. Am J Physiol 272, G885-G893.
28. Chellaiah, A. T., McEwen, D. G., Werner, S., Xu, J., and Ornitz, D. M. (1994). Fibroblast growth factor receptor (FGFR) 3. Alternative splicing in immunoglobulin-like domain III creates a receptor highly specific for acidic FGF/FGF-1. J Biol Chem 269, 11620-11627.
29. Ornitz, D. M., Xu, J., Colvin, J. S., McEwen, D. G., MacArthur, C. A., Coulier, F., Gao, G., and Goldfarb, M. (1996). Receptor specificity of the fibroblast growth factor family. J Biol Chem 271, 15292-15297.
30. Zeeh, J. M., Procaccino, F., Hoffmann, P., Aukerman, S. L., McRoberts, J. A., Soltani, S., Pierce, G. F., Lakshmanan, J., Lacey, D., and Eysselein, V. E. (1996). Keratinocyte growth factor ameliorates mucosal injury in an experimental model of colitis in rats. Gastroenterology 110, 1077-1083.
31. Miceli, R., Hubert, M., Santiago, G., Yao, D. L., Coleman, T. A., Huddleston, K. A., and Connolly, K. (1999). Efficacy of keratinocyte growth factor-2 in dextran sulfate sodium-induced murine colitis. J Pharmacol Exp Ther 290, 464-471.
32. Sandborn, W. J., Sands, B. E., Wolf, D. C., Valentine, J. F., Safdi, M., Katz, S., Isaacs, K. L., Wruble, L. D., Katz, J., Present, D. H., Loftus, E. V., Jr., Graeme-Cook, F., Odenheimer, D. J., and Hanauer, S. B. (2003). Repifermin (keratinocyte growth factor-2) for the treatment of active ulcerative colitis: a randomized, double-blind, placebo-controlled, dose-escalation trial. Aliment Pharmacol Ther 17, 1355-1364.
33. Sperinde, G. V., and Nugent, M. A. (2000). Mechanisms of fibroblast growth factor 2 intracellular processing: a kinetic analysis of the role of heparan sulfate proteoglycans. Biochemistry 39, 3788-3796.
34. Dull, R. O., Dinavahi, R., Schwartz, L., Humphries, D. E., Berry, D., Sasisekharan, R., and Garcia, J. G. (2003). Lung endothelial heparan sulfates mediate cationic peptide-induced barrier dysfunction: a new role for the glycocalyx. Am J Physiol Lung Cell Mol Physiol 285, L986-995.
35. Padera, R., Venkataraman, G., Berry, D., Godvarti, R., and Sasisekharan, R. (1999). FGF-2/fibroblast growth factor receptor/heparin-like glycosaminoglycan interactions: a compensation model for FGF-2 signaling. Faseb J 13, 1677-1687.
36. Berry, D., Shriver, Z., Natke, B., Kwan, C. P., Venkataraman, G., and Sasisekharan, R. (2003). Heparan sulphate glycosaminoglycans derived from endothelial cells and smooth muscle cells differentially modulate fibroblast growth factor-2 biological activity through fibroblast growth factor receptor-1. Biochem J 373, 241-249.
37. Berry, D., Kwan, C. P., Shriver, Z., Venkataraman, G., and Sasisekharan, R. (2001). Distinct heparan sulfate glycosaminoglycans are responsible for mediating Fibroblast Growth Factor-2 biological activity though different Fibroblast Growth Factor Receptors. Faseb J 15, 1422-1424.
38. Anderson, J. M., Van Itallie, C. M., Peterson, M. D., Stevenson, B. R., Carew, E. A., and Mooseker, M. S. (1989). ZO-1 mRNA and protein expression during tight junction assembly in Caco-2 cells. J Cell Biol 109, 1047-1056.
39. Youakim, A., and Ahdieh, M. (1999). Interferon-gamma decreases barrier function in T84 cells by reducing ZO-1 levels and disrupting apical actin. Am J Physiol 276, G1279-G1288.
40. McQuade, K. J., and Rapraeger, A. C. (2003). Syndecan-1 transmembrane and extracellular domains have unique and distinct roles in cell spreading. J Biol. Chem.
41. Farquhar, M. G., and Palade, G. E. (1963). Junctional complexes in various epithelia. Cell Biol 17, 375-412.
42. Gumbiner, B. M. (1996). Cell adhesion: the molecular basis of tissue architecture and morphogenesis. Cell 84, 345-357.
43. Yeaman, C., Grindstaff, K. K., and Nelson, W. J. (1999). New perspectives on mechanisms involved in generating epithelial cell polarity. Physiol Rev 79, 73-98.
44. Matter, K., and Balda, M. S. (2003). Signalling to and from tight junctions. Nat Rev Mol Cell Biol 4, 225-236.
45. Nusrat, A., Turner, J. R., and Madara, J. L. (2000). Molecular physiology and pathophysiology of tight junctions. IV. Regulation of tight junctions by extracellular stimuli: nutrients, cytokines, and immune cells. Am J Physiol Gastrointest Liver Physiol 279, G851-857.
62. Han, X., Fink, M. P., and Delude, R. L. (2003). Proinflammatory cytokines cause NO*-dependent and -independent changes in expression and localization of tight junction proteins in intestinal epithelial cells. Shock 19, 229-237.
47. Hopkins, A. M., Walsh, S. V., Verkade, P., Boquet, P., and Nusrat, A. (2003). Constitutive activation of Rho proteins by CNF-1 influences tight junction structure and epithelial barrier function. J Cell Sci 116, 725-742.
48. Mochizuki, Y., Tsuda, S., Kanetake, H., and Kanda, S. (2002). Negative regulation of urokinase-type plasminogen activator production through FGF-2-mediated activation of phosphoinositide 3-kinase. Oncogene 21, 7027-7033.
49. Bayatti, N., and Engele, J. (2001). Cyclic AMP modulates the response of central nervous system glia to fibroblast growth factor-2 by redirecting signalling pathways. Neurochem 78, 972-980.
50. Fannon, M., and Nugent, M. A. (1996). Basic fibroblast growth factor binds its receptors, is internalized, and stimulates DNA synthesis in Balb/c3T3 cells in the absence of heparan sulfate. J Biol Chem 271, 17949-17956.
51. Rapraeger, A. C., Kruffka, A., and Olwin, B. B. (1991). Requirement of heparin sulfate for bFGF-mediated fibroblast growth and myoblast differentiation. Science 252, 1705-1708.
52. Couchman, J. R. (2003). Syndecans: proteoglycan regulators of cell-surface microdomains? Nat Rev Mol Cell Biol 4, 926-937.
53. Fialka, I., Steinlein, P., Ahorn, H., Bock, G., Burbelo, P. D., Haberfellner, M., Lottspeich, F., Paiha, K., Pasquali, C., and Huber, L. A. (1999). Identification of syntenin as a protein of the apical early endocytic compartment in Madin-Darby canine kidney cells. J Biol Chem 274, 26233-26239.
54. Zimmermann, P., Tomatis, D., Rosas, M., Grootjans, J., Leenaerts, I., Degeest, G., Reekmans, G., Coomans, C., and David, G. (2001). Characterization of syntenin, a syndecan-binding PDZ protein, as a component of cell adhesion sites and microfilaments. Mol Biol Cell 12, 339-350.
55. Grootjans, J. J., Zimmermann, P., Reelmians, G., Smets, A., Degeest, G., Durr, J., and David, G. (1997). Syntenin, a PDZ protein that binds syndecan cytoplasmic domains. Proc Natl Acad Sci USA 94, 13683-13688.
56. Chua, C. C., Rahimi, N., Forsten-Williams, K., and Nugent, M. A. (2004). Heparan sulfate proteoglycans function as receptors for fibroblast growth factor-2 activation of extracellular signal-regulated kinases 1 and 2. Circ Res 94, 316-323.
57. Chu, C. L., Buczek-Thomas, J. A., and Nugent, M. A. (2004). Heparan sulphate proteoglycans modulate fibroblast growth factor-2 binding through a lipid raft-mediated mechanism. Biochem J 379, 331-341.
58. el-Hariry, I., Pagnatelli, M., and Lemoine, N. (1997). Fibroblast growth factor 1 and fibroblast growth factor 2 immunoreactivity in gastrointestinal tumours. J Pathol 181, 39-45.
59. Bryckaert, M., Guillonneau, X., Hecquet, C., Perani, P., Courtois, Y., and Mascarelli, F. (2000). Regulation of proliferation-survival decisions is controlled by FGF1 secretion in retinal pigmented epithelial cells. Oncogene 19, 4917-4929.
60. Brauchle, M., Madlener, M., Wagner, A. D., Angermeyer, K., Lauer, U., Hofschneider, P. H., Gregor, M., and Werner, S. (1996). Keratinocyte growth factor is highly overexpressed in inflammatory bowel disease. Am J Pathol 149, 521-529.
61. Podolsky, D. K. (2002). Inflammatory bowel disease. N Engl J Med 347, 417-429.
62. Gardiner, K. R., Anderson, N. H., Rowlands, B. J., and Barbul, A. (1995). Colitis and colonic mucosal barrier dysfunction. Gut 37, 530-535.
63. Hermiston, M. L., and Gordon, J. I. (1995). Inflammatory bowel disease and adenomas in mice expressing a dominant negative N-cadherin. Science 270, 1203-1207.
64. Spivak-Kroizman, T., Lemmon, M. A., Dikic, I., Ladbury, J. E., Pinchasi, D., Huang, J., Jaye, M., Crumley, G., Schlessinger, J., and Lax, I. (1994). Heparin-induced oligomerization of FGF molecules is responsible fo FGF receptor dimerization, activation, and cell proliferation. Cell 79, 1015-1024.
65. Roghani, M., Mansukhani, A., Dell'Era, P., Bellosta, P., Basilico, C., Rifkin, D. B., and Moscatelli, D. (1994). Heparin increases the affinity of basic fibroblast growth factor for its receptor but is not required for binding. J Biol Chem 269, 3976-3984.
66. Park, P. W., Pier, G. B., Hinkes, M. T., and Bernfield, M. (2001). Exploitation of syndecan-1 shedding by Pseudomonas aeruginosa enhances virulence. Nature 411, 98-102.
67. Li, Q., Park, P. W., Wilson, C. L., and Parks, W. C. (2002). Matrilysin shedding of syndecan-1 regulates chemokine mobilization and transepithelial efflux of neutrophils in acute lung injury. Cell 111, 635-646.
68. Alexander, C. M., Reichsman, F., Hinkes, M. T., Lincecum, J., Becker, K. A., Cumberledge, S., and Bernfield, M. (2000). Syndecan-1 is required for Wnt-1-induced mammary tumorigenesis in mice. Nat Genet. 25, 329-332.
69. Fialka, I., Steinlein, P., Ahorn, H., Bock, G., Burbelo, P. D., Haberfellner, M., Lottspeich, F., Paiha, K., Pasquali, C., and Huber, L. A. (1999). Identification of syntenin as a protein of the apical early endocytic compartment in Madin-Darby canine kidney cells. J Biol Chem 274, 26233-26239.
70. Couchman, J. R. (2003). Syndecans: proteoglycan regulators of cell-surface microdomains? Nat Rev Mol Cell Biol 4, 926-937.
71. Dignass, A. U., Tsunekawa, S., and Podolsky, D. K. (1994). Fibroblast growth factors modulate intestinal epithelial cell growth and migration. Gastroenterology 106, 1254-1262.
72. Hsia, E., Richardson, T. P., and Nugent, M. A. (2003). Nuclear localization of basic fibroblast growth factor is mediated by heparan sulfate proteoglycans through protein kinase C signaling. J Cell Biochem 88, 1214-1225.
73. Farquhar, M. G., and Palade, G. E. (1963). Junctional complexes in various epithelia. Cell Biol 17, 375-412.
74. Sasisekharan, R., Shriver, Z., Venkataraman, G., and Narayanasami, U. (2002). Roles of heparan-sulphate glycosaminoglycans in cancer. Nat Rev Cancer 2, 521-528.
75. Fitzgerald, M. L., Wang, Z., Park, P. W., Murphy, G., and Bernfield, M. (2000). Shedding of syndecan-1 and -4 ectodomains is regulated by multiple signaling pathways and mediated by a TIMP-3-sensitive metalloproteinase. J Cell Biol 148, 811-824.
76. Travis, A. J., Merdiushev, T., Vargas, L. A., Jones, B. H., Purdon, M. A., Nipper, R. W., Galatioto, J., Moss, S. B., Hunnicutt, G. R., and Kopf, G. S. (2001). Expression and localization of caveolin-1, and the presence of membrane rafts, in mouse and Guinea pig spermatozoa. Dev Biol 240, 599-610.
77. Tkachenko, E., and Simons, M. (2002). Clustering induces redistribution of syndecan-4 core protein into raft membrane domains. J Biol Chem 277, 19946-19951.
78. Brodsky; R. A., Mulhina, G. L., Li, S., Nelson, K. L., Chiurazzi, P. L., Buckley, J. T., and Borowitz, M. J. (2000). Improved detection and characterization of paroxysmal nocturnal hemoglobinuria using fluorescent aerolysin. Am J Clin Pathol 114, 459-466.
79. Tkachenko, E., Lutgens, E., Stan, R. V., and Simons, M. (2004). Fibroblast growth factor 2 endocytosis in endothelial cells proceed via syndecan-4-dependent activation of Rac1 and a Cdc42-dependent macropinocytic pathway. J Cell Sci 117, 3189-3199.
80. Nelson, W. J., and Nusse, R. (2004). Convergence of Wnt, beta-catenin, and cadherin pathways. Science 303, 1483-1487.
81. Cadigan, K. M., and Nusse, R. (1997). Wnt signaling: a common theme in animal development. Genes Dev 11, 3286-3305.
82. Ciruna, B., and Rossant, J. (2001). FGF signaling regulates mesoderm cell fate specification and morphogenetic movement at the primitive streak. Dev Cell 1, 37-49.
83. Perez-Moreno, M., Jamora, C., and Fuchs, E. (2003). Sticky business: orchestrating cellular signals at adherens junctions. Cell 112, 535-548.
84. Gumbiner, B. M. (1996). Cell adhesion: the molecular basis of tissue architecture and morphogenesis. Cell 84, 345-357.
85. Yeaman, C., Grindstaff, K. K., and Nelson, W. J. (1999). New perspectives on mechanisms involved in generating epithelial cell polarity. Physiol Rev 79, 73-98.
86. Matter, K., and Balda, M. S. (2003). Signalling to and from tight junctions. Nat Rev Mol Cell Biol 4, 225-236.
87. Blumberg, R. S., and Strober, W. (2001). Prospects for research in inflammatory bowel disease. Jama 285, 643-647.
88. Cheng, C. W., Smith, S. K., and Charnock-Jones, D. S. (2003). Wnt-1 signaling inhibits human umbilical vein endothelial cell proliferation and alters cell morphology. Exp Cell Res 291, 415-425.
89. Katoh, M. (2001). Differential regulation of WNT2 and WNT2B expression in human cancer. Int J Mol Med 8, 657-660.
90. Katoh, M. (2002). Regulation of WNT3 and WNT3A mRNAs in human cancer cell lines NT2, MCF-7, and MKN45. Int J Oncol 20, 373-377.
91. Qiang, Y. W., Endo, Y., Rubin, J. S., and Rudikoff, S. (2003). Wnt signaling in B-cell neoplasia. Oncogene 22, 1536-1545.
92. Numa, F., Hirabayashi, K., Kawasaki, K., Sakaguchi, Y., Sugino, N., Suchiro, Y., Suminami, Y., Hirakawa, H., Umayahara, K., Nawata, S., Ogata, H., and Kato, H. (2002). Syndecan-1 expression in cancer of the uterine cervix: association with lymph node metastasis. Int J Oncol 20, 39-43.
93. Sperinde, G. V., and Nugent, M. A. (2000). Mechanisms of fibroblast growth factor 2 intracellular processing: a kinetic analysis of the role of heparan sulfate proteoglycans. Biochemistry 39, 3788-3796.

Each of the foregoing patents, patent applications and references that are recited in this application are herein incorporated in their entirety by reference. Having described the presently preferred embodiments, and in accordance with the present invention, it is believed that other modifications, variations and changes will be suggested to those skilled in the art in view of the teachings set forth herein. It is, therefore, to be understood that all such variations, modifications, and changes are believed to fall within the scope of the present invention as defined by the appended claims.

I/We claim:

1. A method for altering an inflammatory response, comprising:
(a) contacting one or more cells affected by the inflammatory response with a composition comprising fibroblast growth factor-1 (FGF1) and at least one other fibroblast growth factor (FGF), wherein the composition is in an amount effective to alter the inflammatory response;
(b) contacting one or more cells affected by the inflammatory response with a composition comprising a stabilized FGF dimer, wherein the composition is in an amount effective to alter the inflammatory response, and wherein the inflammatory response is not associated with a wound, a scar, an ulcerating disease, inflammatory neuropathy or chronic inflammatory demyelinating polyradiculoneuropathy; or
(c) contacting one or more cells affected by the inflammatory response with a composition comprising an agent that alters Wnt signaling, wherein the composition is in an amount effective to alter the inflammatory response.

2. The method of claim 1, wherein the at least one other FGF is FGF2, FGF7, FGF10 or FGF20.

3. The method of claim 1, wherein the at least one other FGF is FGF2, FGF7 or both.

4. The method of claim 1, wherein the FGF1 is in a dimeric form.

5. The method of claim 1, wherein the at least one other FGF is in a dimeric form.

6. The method of claim 5, wherein the at least one other FGF in dimeric form is FGF2 in dimeric form.

7. The method of claim 1, wherein the FGF1 and the at least one other FGF are both in a dimeric form.

8. The method of claim 1, wherein the one or more cells affected by the inflammatory response are in a subject, and the composition is administered to the subject.

9. The method of claim 8, wherein the subject has a disease associated with an improper immune response.

10. The method of claim 9, wherein the disease associated with an improper immune response is an inflammatory bowel disease, autoimmune disease, a chronic disease with bouts of acute inflammation, Lyme disease, tuberculosis or multiple myeloma.

11. The method of claim 10, wherein the inflammatory bowel disease is ulcerative colitis or Crohn's disease.

12. The method of claim 8, wherein the subject has exuberant granulomas or keloids.

13. The method of claim 8, wherein the subject is in need of wound healing or scar reduction.

14. The method of claim 8, wherein the subject is in need of cell proliferation or angiogenesis.

15. The method of claim 8, wherein the subject has an ulcer.

16. The method of claim 15, wherein the ulcer is a diabetic ulcer.

17. The method of claim 1, wherein the composition further comprises a pharmaceutically acceptable carrier.

18. The method of claim 1, wherein the composition further comprises an additional therapeutic agent.

19-29. (canceled)

30. A method for altering an inflammatory response in a subject, comprising:
(a) administering to the subject a syndecan agent, wherein the syndecan agent is in an amount effective to alter the inflammatory response, and wherein the syndecan agent is not TMB; or
(b) administering to a subject a composition comprising a FGF in an amount effective to transiently disrupt intercellular junctions.

31-53. (canceled)

54. A composition comprising:
(a) FGF1, at least one other FGF and a pharmaceutically acceptable carrier;
(b) a stabilized FGF dimer, at least one additional therapeutic agent and a pharmaceutically acceptable carrier, wherein the at least one additional therapeutic agent is an anti-inflammatory agent, an anti-cancer agent or an agent for treating an immunologic disorder:
(c) a syndecan agent and a pharmaceutically acceptable carrier, wherein the syndecan agent is not TMB, and wherein the syndecan agent is in an amount effective to alter an inflammatory response; or
(d) an agent that alters Wnt signaling and a pharmaceutically acceptable carrier.

55-98. (canceled)
